(19) United States
(12) Patent Application Publication
(10) Pub. No.: US 2007/0005090 A1
(43) Pub. Date: Jan. 4, 2007

(54) Device and Method for Vascular Tamponade Following Percutaneous Puncture

(76) Inventors: Willet F. Whitmore III, Longboat Key, FL (US); Roger F. Wilson, Sarasota, FL (US)

Correspondence Address: STEPTOE & JOHNSON LLP, 1330 CONNECTICUT AVENUE, N.W., WASHINGTON, DC, 20036, US

(21) Appl. No.: 11/278,660

(22) Filed: Apr. 4, 2006

Related U.S. Application Data
(63) Continuation-in-part of application No. 11/095,586, filed on Apr. 1, 2005.
(60) Provisional application No. 60/559,414, filed on Apr. 2, 2004.
(60) Provisional application No. 60/575,792, filed on May 28, 2004.
(60) Provisional application No. 60/614,593, filed on Oct. 1, 2004.
(60) Provisional application No. 60/667,688, filed on Apr. 4, 2005.

(51) Int. Cl. A61M 29/00

(52) U.S. Cl. 606/191

(57) ABSTRACT

A device for vascular tamponade includes a compression portion and a curvilinear articulating arm coupled to the compression portion. A method for vascular tamponade includes: manually applying a desired pressure proximate a puncture in a vessel by positioning a compression member to apply pressure against skin of a patient, the desired pressure permitting clot formation at the puncture; coupling the compression member to an object disposed in fixed relationship to the puncture; fixing the compression member in a position so that the desired pressure is maintained proximate the puncture without continuing to manually apply the desired pressure.

### CROSS-REFERENCE TO RELATED APPLICATIONS

This application is a continuation-in-part of application Ser. No. 11/095,586 filed Apr. 1, 2005 and entitled “Support System for Use When Performing Medical Imaging of a Patient” which claims the benefits of Provisional Application No. 60/559,414 filed Apr. 2, 2004, Provisional Application No. 60/575,792 filed May 28, 2004, and Provisional Application No. 60/614,593 filed Oct. 1, 2004 under 35 U.S.C. § 119(e), and the entire contents of each of these applications are expressly incorporated herein by reference thereto. In addition, the benefits of Provisional Application No. 60/667,688 filed Apr. 4, 2005 and entitled “Device and Method for Vascular Tamponade Following Percutaneous Puncture” are claimed under 35 U.S.C. § 119(e), and the entire contents of this provisional application are expressly incorporated herein by reference thereto.

### FIELD OF THE INVENTION

The invention relates to a device and method for applying compression to an anatomical region. More particularly, the invention relates to a device and method for vascular tamponade following percutaneous puncture.

### BACKGROUND OF THE INVENTION

Many diagnostic and therapeutic medical procedures are performed via intra-vascular access using a percutaneous approach. The common entry points into the vascular tree are through the Femoral vessels in the groin and through the Axillary vessels in the arm. Both types of vessels allow access to all the major organs for example using thin wires and catheters combined with x-ray fluoroscopy and contrast agents for guidance. Diagnostic angiography and insertion of specialized catheters used therapeutically such as for inserting stents, injecting microspheres or drugs, and placement of occlusive or filtering devices are among the typical procedures done by these routes. At the conclusion of these procedures, a hole that varies in size depending on the procedure is left in the vessel wall at the point of access. This hole must be sealed by natural clotting or invasive methods to prevent serious bleeding. This is especially the case if the hole is in an artery and it is a relatively large hole and if the vessel is compromised by disease. Depending on the size of the hole, how it is managed rests with the judgment of the physician.

Various biologic compounds and devices for closing, plugging or filling a vascular hole, such as a hole in a vein or artery, have been developed and are in clinical use. However, these devices are themselves invasive, pose some additional risk, and are relatively expensive. Most commonly, the holes are managed by external manual compression on the overlying skin and subcutaneous tissues for up to 30 minutes. This gives sufficient time for normally active clotting mechanisms to generate an effective natural clot to plug the hole. Manual fingertip compression is the standard because it is the safest, most easily performed, and always available method. It is also manually controlled and constant monitoring is a given. The technique is simple. Sufficient pressure is applied to stop the bleeding but excessive pressure that occludes the vessel is avoided. This may be monitored by checking for a pulse distal to the compression site.

The manual compression method does require some skill and experience to achieve a safe and consistent force of compression and to maintain this force consistently over about a 30 minute time period. Also, it is more difficult than one might imagine because the compression force required varies from patient to patient, the area needs to be observed and observable, the area requiring compression is small, the vessel itself or the overlying tissues are somewhat mobile, and the angle of optimal compression may not be simply vertical but will vary greatly from patient to patient and procedure to procedure. The overall goal, as stated above, is to apply sufficient focal pressure to prevent bleeding through the hole without occluding the vessel, and to apply this pressure for a sufficient length of time for a solid plug of fibrin clot to form and seal the hole.

One downside of the manual compression method is that it fully occupies a nurse or technician preventing them from doing anything else for the length of time during which compression is applied. Thus, expensive personnel are occupied for a dull, simple, repetitive and somewhat tiring task.

Problems associated with manual compression have been addressed by several different mechanical devices that more or less mimic the manual compression method. One non-invasive device available from Radi Medical Systems AB is called FemoStop® and is used in femoral artery punctures at the groin area. This device wraps around the hips and provides a rigid backstop over the puncture site. A balloon is inflated between the backstop and the puncture site to compress the soft tissues overlying the puncture site. The balloon is inflated until compression is sufficient. Initially, the artery may be compressed to complete occlusion for several minutes and then the pressure, monitored by a gauge connected to the balloon, typically is reduced until there is a pulse felt in the artery distal to the balloon. This device can be cumbersome to set up, covers the puncture site from view in an undesirable manner, and blocks normal tactile feedback. One advantage to this system is that it will not be easily dislodged from an effective position by minor patient movement.

Another device offered by Advanced Vascular Dynamics is called the CompressAR® System. It uses a simple mechanical post that is mounted on a plate that is held in position underneath a mattress/patient by the weight of the patient. A vertically sliding cantilevered bar is used to hold a disposable plastic self-aligning disk against the groin to achieve suitable compression. This device also can be cumbersome to set up, interferes with the normal manual approach to vessel compression by blocking tactile feedback in the critical area, and is difficult to position and adjust. It also compresses a larger area than is considered ideal. However, this device does allow better visibility of the wound than the FemoStop device.

### SUMMARY OF THE INVENTION

The present invention relates to a non-invasive device and method for applying external compression to a punctured vessel. This device may allow full use of the normal tactile manual method of fingertip manual compression, by placing a mechanical pad between the fingertips and the skin that has a hole in a central area. This hole gives direct skin access for palpating the vessel for location and pulse. In an exemplary embodiment, the anatomical site to be compressed is otherwise covered by the pad, the desired pressure and angle of compression is achieved, and a mechanical arm is brought in to engage the mechanical pad and locked in position. The mechanical arm may be attached to a fixed base which in turn may be attached to any of a wide variety of fixed surfaces or objects without moving or displacing the patient. Complete freedom of movement of the end of the arm may be available to engage the mechanical pad at the proper location and angle of compression. The arm may be locked in this position and the pressure may be adjusted by an in-column micro-adjust screw mechanism to dial-in an optimum pressure while an operator is continuously palpating the vessel of concern. The fingertip pressure and hand then may be removed while the original force and angle of compression are maintained. The central hole then may be filled to maintain a smooth and uniform compression surface.

In one preferred exemplary embodiment, the mechanical pad connects to a vertical stem that fits into a socket in the mechanical arm and this socket includes the in-line micro-adjust screw mechanism. The central hole in the mechanical pad may be larger than the tip of a middle finger. The pad may be centered over the vessel by palpation with the middle finger while pressure is applied with all three fingers. The first and fourth finger rest on the mechanical pad while the vessel is felt (palpated) with the middle finger. This allows the important tactile feedback required to locate the vessel and apply an appropriate pressure based on experience. The force applied manually is then taken up by the mechanical arm set at an initial locked position and adjustment allowed by the micro-adjust screw. When ideal pressure is achieved as determined by palpation on the vessel, recording the pulse distal to the puncture site, experience of the user, and direct observation of the puncture site, hands are removed and routine procedures for observation and pressure release are followed. This device may allow the user to increase or decrease pressure on the anatomical site by using the micro-adjust screw at any time. The hole for the middle finger may be plugged to complete the compression surface by advancing a secondary surface plug when the fingertips are removed.

Alternative exemplary embodiments may include the addition of relative pressure measuring devices placed in the pressure path to indicate a stable or reproducible setting of the pressure. A spring scale or more complex arrangement such as a piezo-electric digital or hydraulic indicator may be used. Also, in addition to the in line screw mechanism for pressure adjustment, other in line or offset mechanical or hydraulic methods may be used to adjust the pressure force in a finely controlled manner after the mechanical arm is locked if desired.

Another exemplary preferred embodiment may be achieved by temporary or fixed placement in the central area of the compression pad of an ultrasound transducer that is intended to image the underlying vessel. Using the ultrasound image may allow the user to correctly place and orient the compression surface over the desired vessel, to visualize flow through the vessel and to observe any leakage from the puncture site. All this may be done in real time and continuous ultrasound imaging may function as a monitor of performance during the compression period. This could be accomplished using a purpose built transducer that has a proper shape and surface for compression as an integral part of the casing/handle. Alternatively, an ideally shaped surface may be configured to receive (i.e. may function as a shoe) a more generic linear transducer that functions in the optimal frequency range. This shoe may be cleaned and sterilized for re-use or may be a single use disposable pre-sterilized item. A mechanical arm for holding the compression may be configured to securely grasp the ultrasound transducer and a mechanism may be included for micro-adjustment in the direction of the compression force similar to a purely manual embodiment. By using an ultrasound transducer as an integral part of the compression system, optimal results may be achieved. This is because the absolute minimum compression required to prevent leakage may be “dialed-in” using real time confirmation from the direct image, and any change in status may be immediately observed.

Thus, the invention relates to a device for vascular tamponade including a compression portion and a curvilinear articulating arm coupled to the compression portion. The compression portion may be demountably coupled to a body portion and may be slidably associated with the body portion. Also, the compression portion may be spring-loaded. The compression portion may include a first portion with a through hole therein and a second portion for mating with the hole. In some embodiments, the compression portion is formed at least in part by a portion of an ultrasound transducer. The device may further include a bracket, wherein the ultrasound transducer is retained in the bracket. The bracket may be coupled to a base portion and linearly moveable with respect thereto. The base portion may have a linear screw and the bracket may have a boss, and the linear screw may be threadably associated with the boss.

The invention also relates to a device for vascular tamponade including a bracket, a transducer coupled to the bracket, and a curvilinear articulating arm coupled to the bracket. The bracket may include a silicone window disposed proximate an imaging region of the transducer. In some embodiments, the bracket may include a recessed portion for receiving an imaging portion of the transducer and a clamping portion for securing a different portion of the transducer. The bracket may be coupled to a base portion and constrained to linear movement with respect thereto.

In addition, the invention relates to a method for vascular tamponade including: manually applying a desired pressure proximate a puncture in a vessel by positioning a compression member to apply pressure against skin of a patient, the desired pressure permitting clot formation at the puncture; coupling the compression member to a curvilinear articulating arm; fixing the curvilinear articulating arm in a position so that the desired pressure is maintained proximate the puncture without continuing to manually apply the desired pressure. The method may further include monitoring the puncture using ultrasound imaging, wherein the compression member comprises an ultrasound transducer. In some embodiments, the ultrasound transducer may be disposed in a sterile sheath. In some embodiments, the ultrasound transducer may be separated from the skin by a separate layer of silicone. The method may further include monitoring the desired pressure and/or measuring the desired pressure. The desired pressure may be applied by the compression member prior to coupling the compression member to the curvilinear articulating arm.

Furthermore, the invention relates to a method for vascular tamponade including: manually applying a desired pressure proximate a puncture in a vessel by positioning a compression member to apply pressure against skin of a patient, the desired pressure permitting clot formation at the puncture; coupling the compression member to an object disposed in fixed relationship to the puncture; fixing the compression member in a position so that the desired pressure is maintained proximate the puncture without continuing to manually apply the desired pressure. The object may be a rail associated with a bed.

### BRIEF DESCRIPTION OF THE DRAWINGS

Preferred features of the present invention are disclosed in the accompanying drawings, wherein:

FIGS. 1A-1C show a first embodiment of a vascular compression device according to the present invention including (1A) a perspective view thereof, (1B) a side view thereof, and (1C) a perspective view of a compression portion thereof;

FIGS. 1D-1F show a second embodiment of a vascular compression device according to the present invention including (1D) a side view thereof, (1E) a partial cross-sectional side view thereof, and (1F) a partial side view thereof;

FIGS. 1G-1M show additional embodiments of a vascular compression device according to the present invention including (1G) a perspective view of a vascular compression device, (1H) a side view of a portion of the device of FIGS. 1G, (1I) a partial cross-sectional side view of a device similar to FIGS. 1G-1H but with a different shaped knob and a different arrangement of fixed and movable pins, (1J) a partial perspective view of the device of FIGS. 1G, (1K) another partial perspective view of the device of FIGS. 1G, (1L) a partial perspective view of the device of FIG. 1G with a modified body and connection to a coupling portion, and (1M) another partial perspective view of the device of FIG. 1G with a modified body and connection to a coupling portion;

FIG. 1N shows a perspective view of a patient table with a curvilinear articulating arm coupled thereto and a vascular compression device coupled to the arm;

FIGS. 2A-2G show another embodiment of a vascular compression device according to the present invention including (2A) a side perspective view, (2B) a top perspective view, (2C) a bottom perspective view, (2D) a side view, (2E) another side view, (2F) a perspective view of a bracket thereof, and (2G) another perspective view of the bracket thereof;

FIGS. 3A-3G show another embodiment of a vascular compression device according to the present invention including (3A) a side perspective view, (3B) another side perspective view, (3C) a first side view, (3D) a partial cross-sectional side view, (3E) a perspective view of a base thereof, (3F) a perspective view of a drive mechanism thereof, (3G) a side perspective view of the device of FIG. 3A with a different transducer therewith;

FIGS. 4A-4C show the curvilinear articulating arm assembly including (4A) a perspective view, (4B) a partial cross-sectional perspective view, and (4C) a partial side view;

FIGS. 4D-4L show the base handle of FIG. 1, including (4D) a first side view, (4E) a second side view, (4F) a partial perspective view of a first set of components thereof, (4G) a partial side view of a second set of components thereof, (4H) another partial side view of the second set of components thereof (4I) a front view, (4J) a back view, (4K) a top view, and (4L) a bottom view;

FIG. 4M shows a perspective view of a rail clamp for use with the present invention;

FIGS. 4N-4T show the free handle of FIGS. 4A-4C, including (4N) a first side perspective view showing a portion of a tensioning wire therewith, (4-O) a second side perspective view, (4P) a partial perspective view showing a first set of components thereof (4Q) a front perspective view, (4R) a back perspective view, (4S) a top perspective view, and (4T) a bottom perspective view;

FIG. 4U shows a side perspective view of the interface lock of the free handle of FIGS. 4N-4T;

FIG. 5 shows a perspective view of a support system according to the present invention; and

FIGS. 6A-6C show the tray of FIG. 5, including (6A) a top view, (6B) a cross-section taken perpendicular to the central axis of the tray, and (6C) a partial cross-section showing detail taken at VIC.

### DETAILED DESCRIPTION OF THE PREFERRED EMBODIMENTS

Words of orientation as used herein such as “front,” “back” and “top” are used for exemplary convenience only as non-limiting examples of the orientation of features and are not intended to have any particular limiting effect.

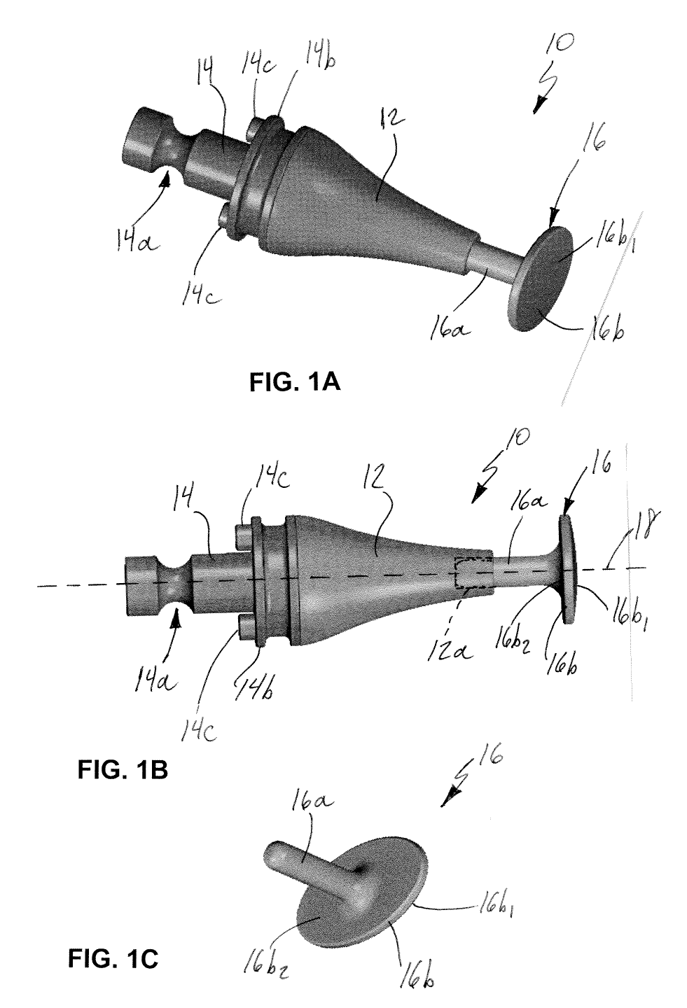

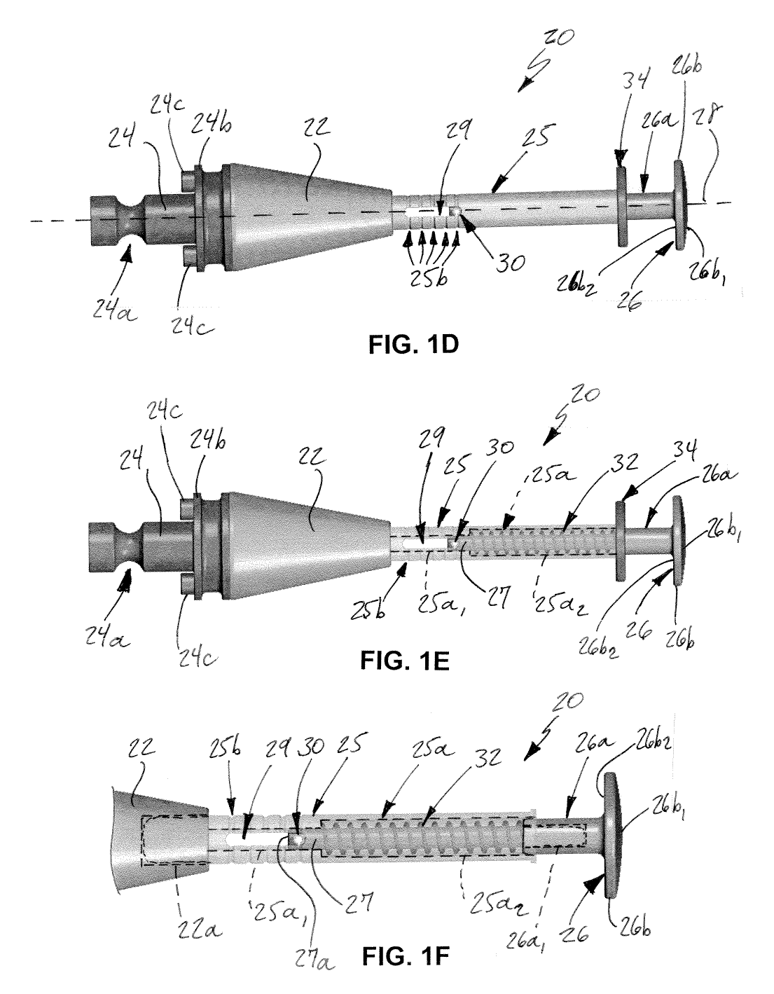

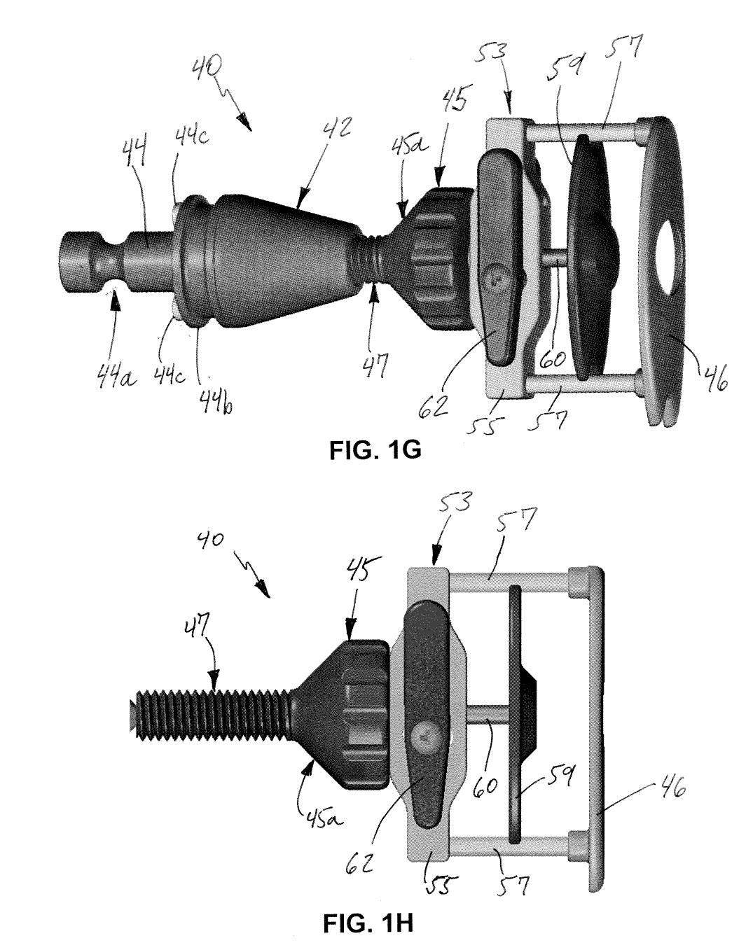

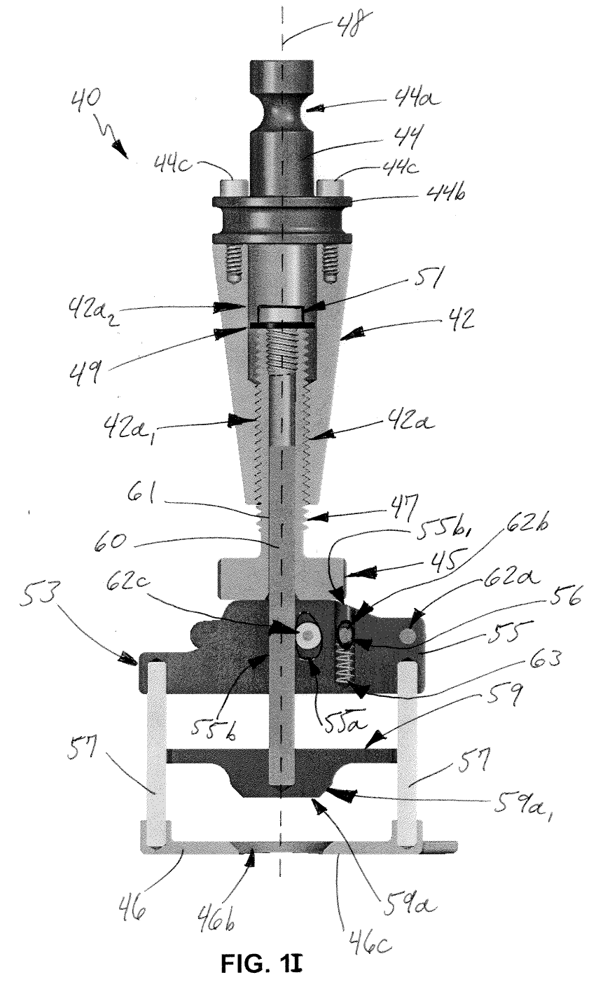

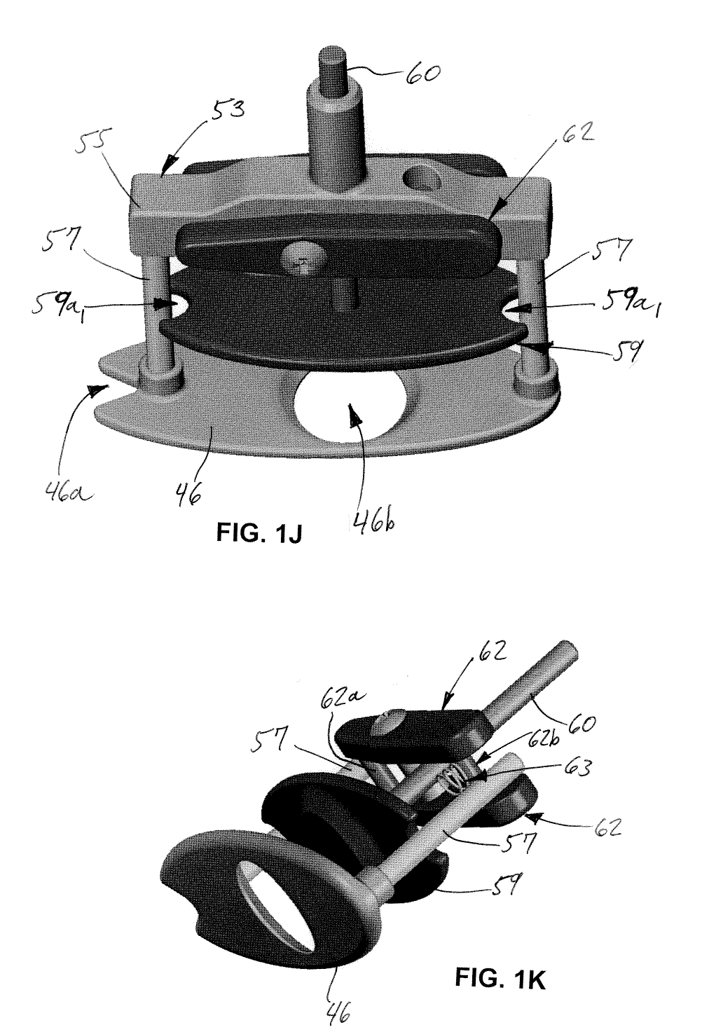

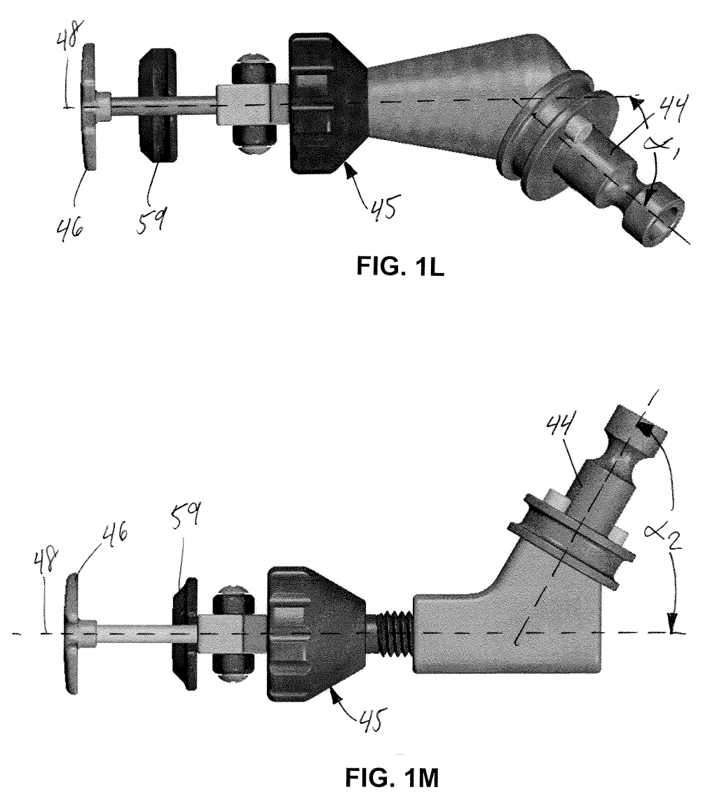

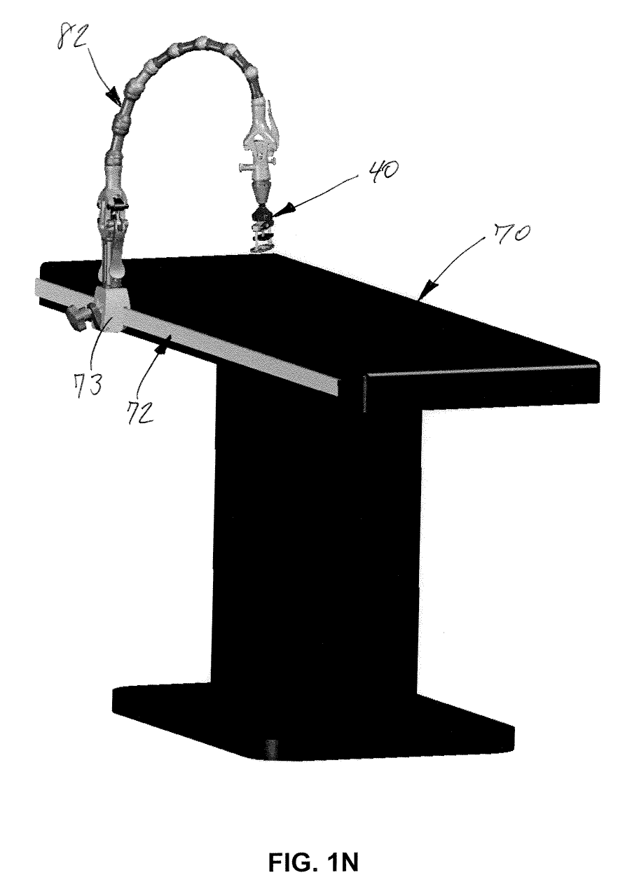

Referring initially to FIGS. 1A-1C, an exemplary embodiment of a vascular compression device 10 according to the present invention is shown. Device 10 includes a body 12 with a coupling portion 14 at a first free end thereof. Coupling portion 14 optionally may include a circumferential groove 14a therein. Body 12 may be demountably attached to coupling portion 14 proximate base portion 14b using screws 14c. Coupling portion 14 preferably is configured to be coupled to an articulating arm assembly as will be described later. A compression portion 16 is demountably coupled to a second free end of body 12. Compression portion 16 includes a stem 16a that is configured and dimensioned to be received and secured for example by friction fit in a hole 12a in body 12. In some exemplary embodiments, stem 16a is received in hole 12a without any friction fit and thus these components may freely move with respect to one another except when compressed against each other when device 10 is used to exert pressure against an anatomical region. Portion 16 also includes a compression region 16b which for example may be a generally circular region disposed in a plane perpendicular to central axis 18 of device 10. Compression region 16b has front and back faces 16b₁ and 16b₂, respectively, and front face 16b₁ for example may be a portion of a sphere and thus be arcuate and symmetrical about central axis 18.

In use, compression region 16b may directly contact a patient proximate the anatomical region of interest for vascular tamponade. Portion 16 may be provided as a sterile component that may be disposable and gauze pads optionally may be placed along compression region 16b so that the patient is directly contacted with thin padding. In alternate embodiments, a sterile sleeve or cover may be provided around a portion or all of device 10. Body 12 and coupling portion 14 of device 10 initially may be secured to an articulating arm assembly as will be described, which for example may be mounted to the railing of a hospital bed or otherwise fixed to a stable object. The operator of device 10 may first position compression portion 16 proximate the anatomical region of interest by squeezing stem 16a between their fingers of one hand and positioning compression region 16b (with or without gauze thereon) on the patient's skin over an entry wound or hole in a vessel and apply a desired amount of pressure. With a second hand, the operator may then grasp and maneuver body 12 so that stem 16a of compression portion 16 is received in hole 12a of body 12. Stem 16a may be advanced in hole 12a until it “bottoms out,” and in one exemplary embodiment a portion of stem 16a extends from a free end of body 12 when stem 16a is disposed in hole 12a. Once compression portion 16 is held in a desired orientation and position to provide a desired pressure on the hole in the vessel, the articulating arm may be locked in position as will be further described so that compression region 16b remains in place as set by the operator to provide generally constant pressure for a desired period of time. The fingers of the first hand may be removed from holding compression portion 16 so that manual compression is no longer applied by the operator.

Turning to FIGS. 1D-1F, another exemplary embodiment of a vascular compression device 20 according to the present invention is shown. Device 20 includes a body 22 with a coupling portion 24 at a first free end thereof. Coupling portion 24 optionally may include a circumferential groove 24a therein. Body 22 may be demountably attached to coupling portion 24 proximate base portion 24b using screws 24c. Coupling portion 24 preferably is configured to be coupled to an articulating arm assembly as will be described later. A cylinder 25 is configured and dimensioned to be received and secured for example by friction fit in a hole 22a in body 22. Cylinder 25 includes a longitudinal hole 25a therein disposed along central axis 28. Hole 25a includes a first portion 25a₁ having a first diameter and a second portion 25a₂ having a second diameter with the second diameter being greater than the first diameter. A central shaft 27 is disposed on axis 28 and received in hole 25a, and shaft 27 is slidably associated with first portion 25a₁. Cylinder 25 also includes a through slot 29 within which a portion of shaft 27 may be viewed. A pin 30 extends perpendicular to axis 28 through shaft 27 proximate a first free end 27a thereof and rides in opposing sides of slot 29 so that movement of shaft 27 in hole 25a is constrained to linear movement along axis 28 without rotation of shaft 27 about axis 28. A spring 32 is disposed about shaft 27 in second portion 25a₂ of hole 25a.

A scale 25b may be visible on the outer surface of cylinder 25 and may be disposed proximate slot 29. In one embodiment, scale 25b may be in the form of grooves for example disposed circumferentially about cylinder 25 in planes perpendicular to axis 28. In alternate embodiments, other indicia may be used such as other markings including numbers and/or symbols and/or text, or the other indicia may be raised regions on cylinder 25. Preferably, scale 25b indicates evenly spaced positions of pin 30 in slot 29.

A finger pad 34 abuts a free end of cylinder 25. In addition, a compression portion 26 is demountably coupled to a free end of shaft 27 opposite the free end proximate pin 30. Compression portion 26 includes a stem 26a, and a portion of shaft 27 is received in a hole 26a₁ in stem 26a as shown for example in FIG. 1F in which finger pad 34 is not shown. The free end of stem 26a abuts a free end of spring 32 and is configured and dimensioned to be slidably associated with second portion 25a₂ of hole 25a in cylinder 25.

Portion 26 also includes a compression region 26b which for example may be a generally oval-shaped region disposed in a plane perpendicular to central axis 28 of device 20. Compression region 26b has front and back faces 26b₁ and 26b₂, respectively, and front face 26b₁ for example may be a portion of a sphere and thus be arcuate and symmetrical about central axis 28.

Thus, in use, compression portion 26 is spring-loaded. Device 20 allows full use of the normal tactile manual method of fingertip manual compression, but places a mechanical pad in the form of compression portion 26 between the operator's fingertips and the skin. Once the desired anatomical area for compression is covered by front face 26b₁ of compression region 26b of portion 26, and the desired pressure and angle of compression are achieved, an articulating arm as will be described herein may be positioned to engage coupling portion 24 and the arm is then locked in position. The fingertip pressure and hand initially holding portion 26 may then be removed while the original force and angle of compression are maintained. Scale 25b serves as a relative pressure force indicator that may be observed at any time by the operator. Thus, the force applied manually may be observed as a relative position on scale 25b prior to engaging the mechanical articulating arm and then this same force as measured by this relative position may be observed at any time by looking at this reference after the mechanical arm is engaged and the hand is removed from compression portion 26.

During use, the fingertips of the operator preferably are placed on finger pad 34, and pad 34 initially is spaced from compression region 26b. Compression spring 32 preferably provides pressure to resist movement of compression region 26b on the patient. In one embodiment, compression region 26b and pad 34 do not rotate relative to one another. In an exemplary preferred embodiment, a force between about 2 lbs. and about 8 lbs. pounds is indicated by scale 25b and applied to the anatomical region of interest by compression region 26b, although scale 25b only may provide reference marks. In one exemplary embodiment, when the spacing of pad 34 and compression region 26b is at a minimum, 8 lbs. of force is applied by compression region 26b and pin 30 is disposed in slot 29 at an end thereof proximate body 22. In general, the desired force is determined manually and the markings such as rings 25b may be used as a reference to continuously confirm that the mechanical articulating arm has achieved and is maintaining the same desired force that was being applied by compression region 26b using the fingertips of the operator.

In use, compression region 26b may directly contact a patient proximate the anatomical region of interest for vascular tamponade. Portion 26 may be provided as a sterile component that may be disposable and gauze pads optionally may be placed along compression region 26b so that the patient is directly contacted with thin padding. In alternate embodiments, a sterile sleeve or cover may be provided around a portion or all of device 20.

The articulating arm assembly to which device 20 is coupled for example may be mounted to the railing of a hospital bed or otherwise fixed to a stable object that is disposed in a fixed relationship to the patient.

Once compression portion 26 is held in a desired orientation and position to provide a desired pressure on the hole in the vessel, the articulating arm may be locked in position as will be further described so that compression region 26b remains in place as set by the operator to provide generally constant pressure for a desired period of time.

In alternate embodiments, device 20 may include different relative pressure measuring devices such as piezo-electric digital or hydraulic indicators. In addition, vernier type or other mechanical or hydraulic methods may be used to adjust the pressure force in a finely controlled manner after the articulating arm is locked.

Referring next to FIGS. 1G-1N, another exemplary embodiment of a vascular compression device 40 according to the present invention is shown. Device 40 includes a body 42 with a coupling portion 44 at a first free end thereof. Coupling portion 44 optionally may include a circumferential groove 44a therein. Body 42 may be demountably attached to coupling portion 44 proximate base portion 44b using screws 44c. Coupling portion 44 preferably is configured to be coupled to an articulating arm assembly as will be described later. A knob 45 is attached to a threaded shaft 47 which is threadably received in a hole 42a in a threaded portion 42a₁ thereof in body 42 along central axis 48. Knob 45 optionally may include a tapered portion 45a and may be formed of unitary construction with shaft 47. Internally of body 42, shaft 47 is provided with a washer 49 at a free end thereof, and washer 49 is secured to shaft 47 with a bolt 51. Washer 49 is configured and dimensioned to be slidably associated with unthreaded portion 42a₂ of hole 42a in body 42 and to serve as a stop for preventing further travel of shaft 47 when washer 49 reaches the interface between portions 42a₁, 42a₂.

A bracket portion 53 abuts knob 45 and includes a main bracket body 55 and a pair of preferably parallel extensions 57 extending therefrom. Extensions 57 are coupled to main bracket body 55 proximate first free ends thereof and are coupled to a compression portion 46 proximate second free ends thereof. In a preferred exemplary embodiment, compression portion 46 is generally oval-shaped and includes a cut-out 46a disposed along a major axis thereof. Preferably, compression portion 46 includes a central hole 46b therein. A secondary compression portion 59 is disposed between extensions 57 and also may be generally oval-shaped and include cut-outs 59a₁ disposed along a major axis thereof. Portion 59 is coupled to a rod 60 that is disposed along axis 48 and slidably received in a central hole 61 in shaft 47 and a central hole 55b in bracket body 55. Portion 59 includes a central raised portion 59a that is configured and dimensioned to be seated in hole 46b of compression portion 46 to fill the hole. In one exemplary embodiment, hole 46b is tapered while central raised portion 59a includes a like tapered outer circumference 59a₁, so that when portion 59 is positioned proximate portion 46, central raised portion mates with hole 46b to provide a generally complete face 46c for bearing against a desired anatomical region.

Bracket portion 53 includes a pair of levers 62 connected by a fixed pin 62a which serves as a pivot and a movable pin 62b extending through bracket body 55 within a slot 56. Movable pin 62b also extends within and generally perpendicular to hole 55b₁ in bracket body 55 and is spring-loaded by a spring 63 disposed in hole 55b₁. Slot 56 communicates with hole 55b₁. A bushing 62c connected to levers 62 extends through an oblong-shaped hole 55a in bracket body 55 that communicates with central hole 55b in bracket body 55 so that when levers 62 are manipulated by an operator to be generally disposed in a direction transverse to compression portion 46, bushing 62c is urged away from rod 60 to permit movement thereof. Because of the biasing provided by spring 63, the mechanism is self-locking with bushing 62c bearing against rod 60 when levers 62 are generally disposed in a direction parallel to compression portion 46. Thus, levers 62 are operated to loosen contact and readily permit movement of portion 59.

In use, knob 45 and threaded shaft 47 provide a micro-adjust screw mechanism for fine-tuning of pressure applied by compression portions 46, 59.

Preferably, central hole 46b in compression portion 46 is larger than the tip of the middle finger of an operator of device 40. Compression portion 46 may be centered by palpation with the middle finger while pressure is applied to compression portion 46 with three fingers. The first and fourth finger may rest on compression portion 46 while the vessel is felt (palpated) with the middle finger. This allows the important tactile feedback required to locate the vessel and apply an appropriate pressure based on experience. The force applied manually is then taken up by an articulating arm disposed in an initial locked position and adjustment allowed by the micro-adjust screw mechanism. When desired pressure is achieved as determined by palpation on the vessel, recording the pulse distal to the puncture site, experience of the user and direct observation of the puncture site, hands are removed and routine procedures for observation and pressure release are followed. Device 40 allows the user to increase or decrease pressure on the anatomical site of puncture by using the micro-adjust screw mechanism at any time. Hole 46b for the middle finger may be plugged to complete the compression surface by advancing central raised portion 59a of secondary compression portion 59 to be disposed therein.

In alternate embodiments, relative pressure measuring devices may be placed in the pressure path to indicate a stable or reproducible setting of the pressure.

Such devices, for example, could be a spring scale or a piezo-electric digital or hydraulic indicator. Also, in addition to the in-line screw mechanism for pressure adjustment, other in-line or offset mechanical or hydraulic methods may be used to adjust the pressure in a finely controlled manner.

As shown for example in FIGS. 1I, 1L, and 1M, coupling portion 44 may be disposed at a variety of angles with respect to axis 48. In FIG. 1I, coupling portion 44 is aligned with axis 48. In FIG. 1L, coupling portion 44 is disposed at an angle α₁ of about 45° with respect to axis 48, while in FIG. 1M coupling portion 44 is disposed at an angle α₂ of about 60° with respect to axis 48. Thus, coupling portion 44 may be disposed at angles of between about 0° and about 90° with respect to axis 48.

As shown in FIG. 1N, a patient support table 70 with a railing 72 has an articulating arm 82 coupled thereto. Although device 40 is shown coupled to arm 82 at coupling portion 44, the other vascular compression devices disclosed herein also may be coupled to arm 82 as shown.

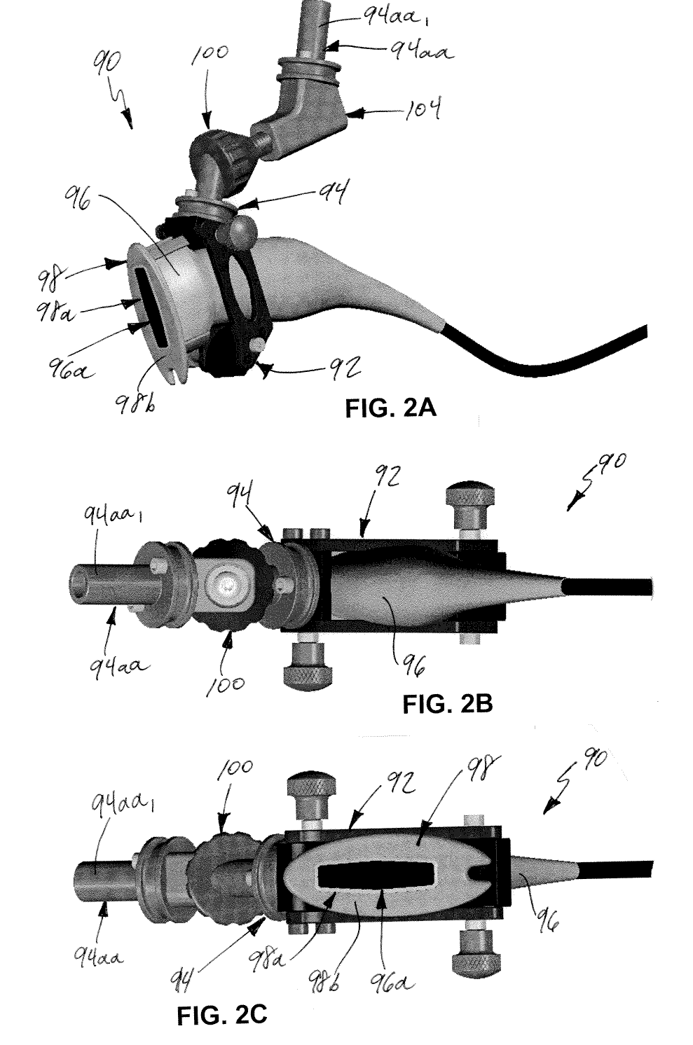

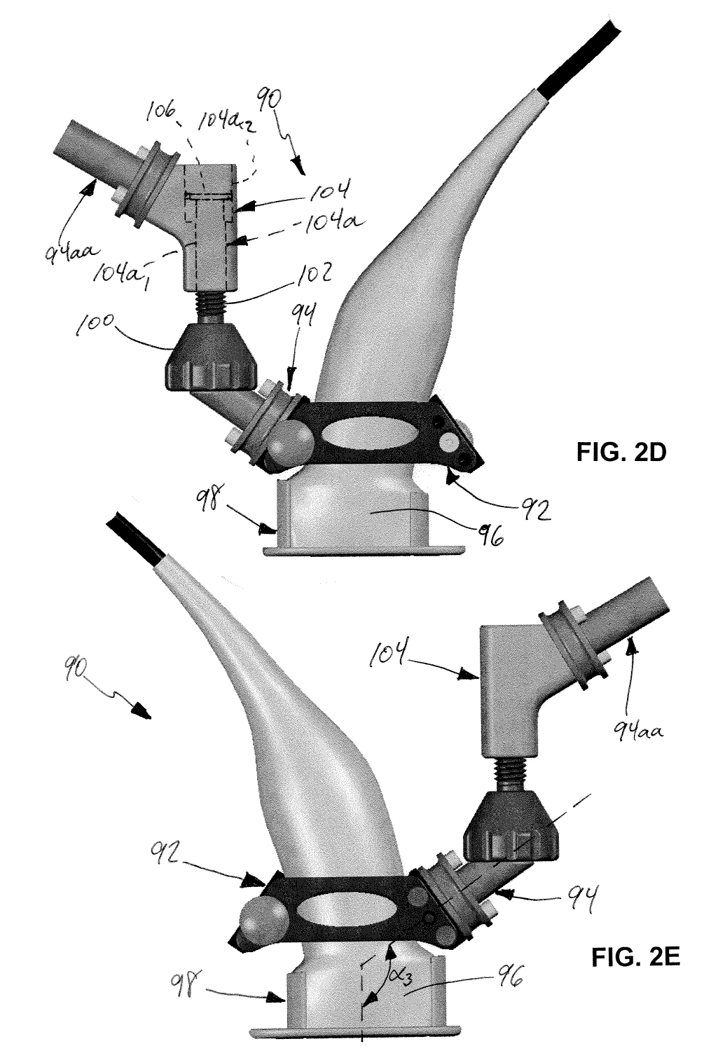

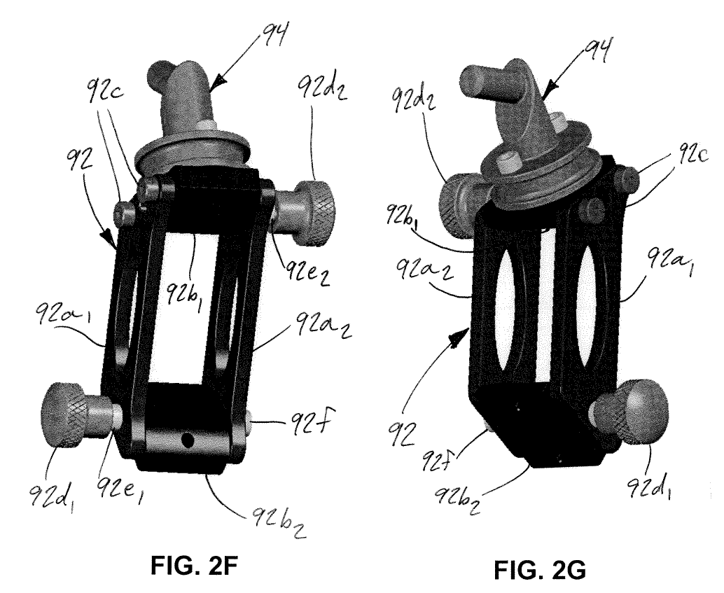

Turning next to FIGS. 2A-2G, yet another exemplary embodiment of a vascular compression device 90 according to the present invention is shown. Device 90 includes a bracket or shoe 92 formed by a pair of generally parallel bracket plates 92a₁, 92a₂ spaced from one another by wedges 92b₁, 92b₂. Plate 92a₁ is coupled to a wedge 92b₁ with a pair of shoulder screws 92c and is further coupled to wedge 92b₂ with a thumb screw 92d₁ and washer 92e₁. Plate 92a₂ is coupled to wedge 92b₁ with a thumb screw 92d₂ and washer 92e₂ and further is coupled to wedge 92b₂ with a shoulder screw 92f. Thumb screws 92d₁ and 92d₂ permit tightening of shoe 92 around an object, as will be described. While wedge 92b₁ is not permitted to angulate, wedge 92b₂ is permitted to angulate and thus swivel about an axis defined by the shafts of thumb screw 92d₁ and shoulder screw 92f which are coaxially disposed. A coupling portion 94 may be demountably coupled to shoe 92 with screws 94c extending from coupling portion 94 into fixed wedge 92b₁. Coupling portion 94 preferably is configured to be coupled to an articulating arm assembly as will be described later.

A linear, ultrasound transducer 96 may be secured in shoe 92, which for example may be formed of plastic, such that the face 96a thereof may be fully exposed through shoe 92. A cover 98 may be provided proximate the imaging end of transducer 96 and may snap-fit thereto with a window 98a configured and dimensioned to permit face 96a of ultrasonic transducer 96 to fit therein and form a generally continuous compression surface with surface 98b of cover 98 for placement adjacent an anatomical region with a puncture.

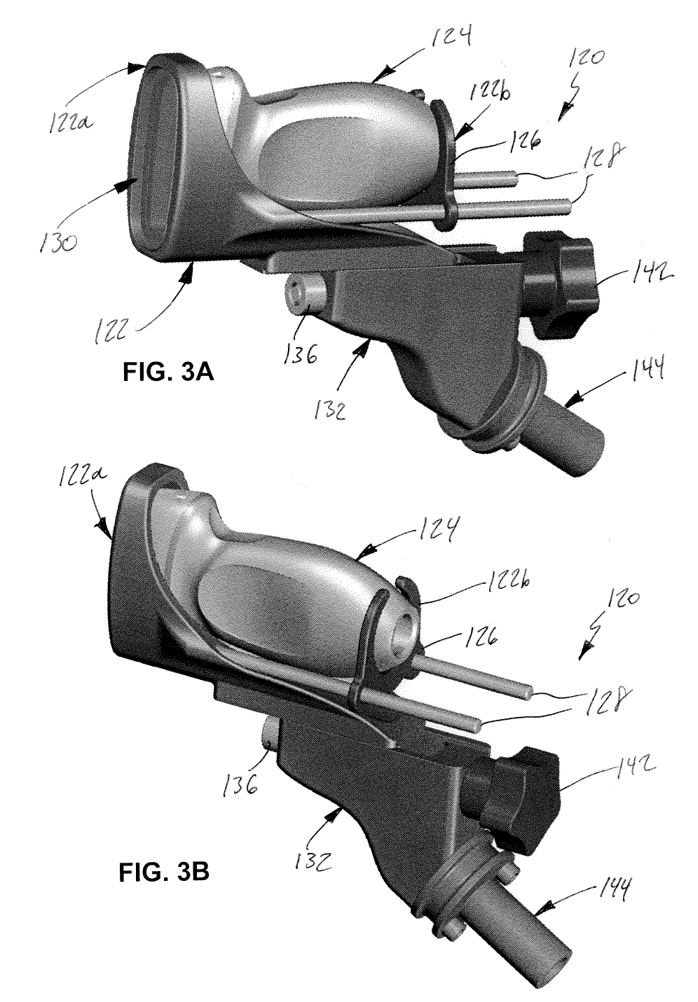

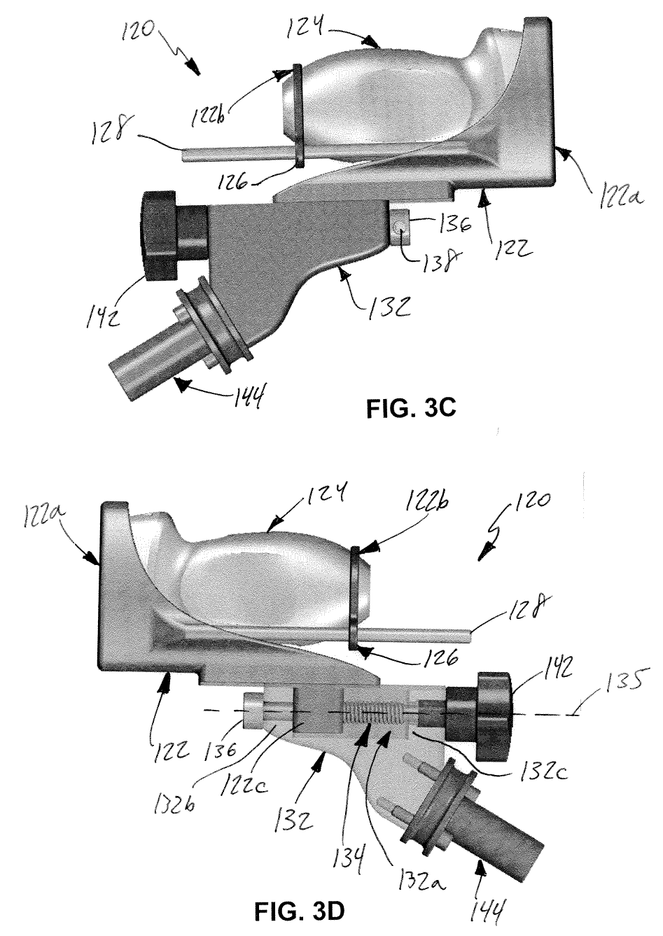

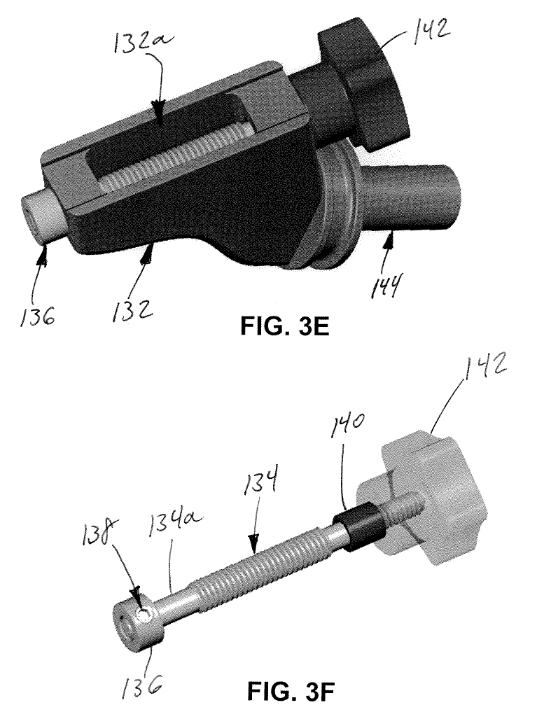

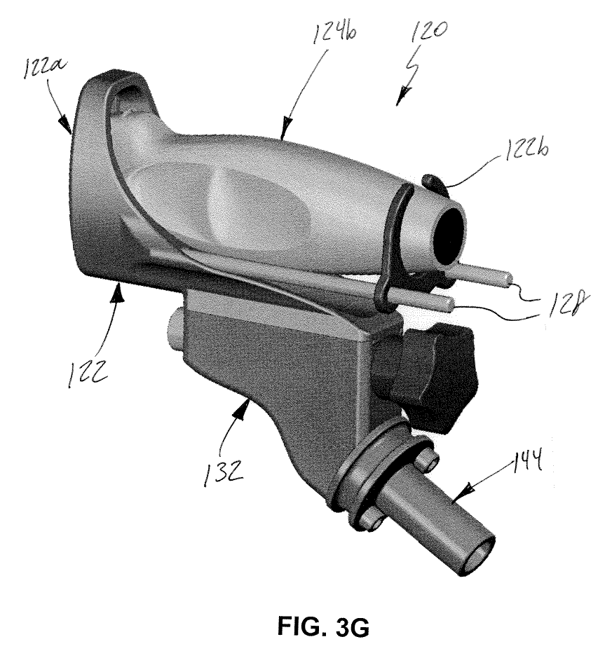

Coupling portion 94 also may include a knob 100 that is attached to a threaded shaft 102 which in turn is threadably received in a hole 104a in a threaded portion 104a₁ thereof in body 104 along central axis 48. Hole 104a further includes an unthreaded portion 104a2, and the difference in diameters of portions 104a₁, 104a₂ forms a shoulder which limits the travel of body 104 on shaft 102 due to head portion 106. An additional coupling portion 94aa may be secured to body 104 for coupling to a curvilinear articulating arm as shown for example in FIG. 3H with respect to device 40.

Shoe 92 for example may be disposable, and a disposable transducer cover may be provided for sterility.

In an exemplary embodiment, an angle α₃ of about 60° may be formed between a plane perpendicular to array 96a and a longitudinal axis defined by coupling portion 94.

In use, ultrasound transducer 96 and optionally cover 98 may serve as a compression region that may directly contact a patient proximate the anatomical region of interest for vascular tamponade. Coupling portion 94aa of device 90 initially may be secured to an articulating arm assembly as will be described, which for example may be mounted to the railing of a hospital bed or otherwise fixed to a stable object. The operator of device 90 may first position transducer 96 secured in shoe 92 proximate the anatomical region of interest on the patient's skin over an entry wound or hole in a vessel and apply a desired amount of pressure. With a second hand, the operator may then couple device 90 to an articulating arm. Once transducer 96 is held in a desired orientation and position to provide a desired pressure on the hole in the vessel, the articulating arm may be locked in position as will be further described so that transducer 96 remains in place as set by the operator to provide generally constant pressure for a desired period of time. The puncture and healing thereof may be monitored by an operator using ultrasound images from transducer 96.

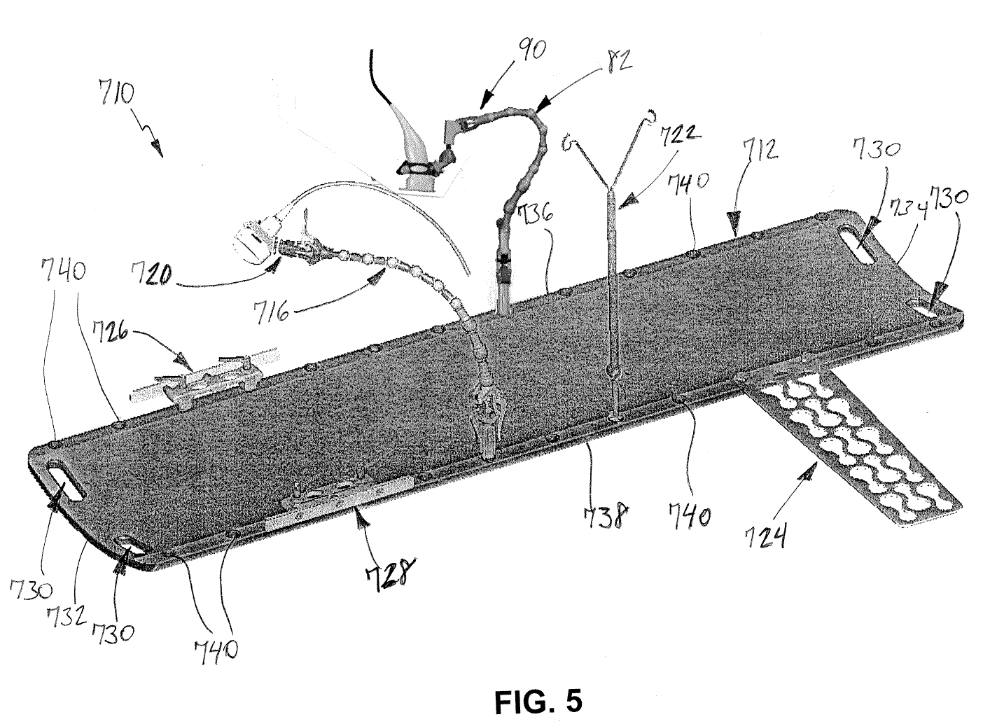

Referring next to FIGS. 3A-3G, yet another exemplary embodiment of a vascular compression device 120 according to the present invention is shown. A bracket or shoe 122 is configured and dimensioned to receive an ultrasound transducer 124 which is held or cradled between a front portion 122a shaped to house the face of, or entire linear crystal array of transducer 124 and a back portion 122b in the form of a movable clip 126. Shoe 122 further includes a pair of generally parallel rods 128 on which clip 126 is slidably mounted with sufficient friction to prevent undesired movement thereon when transducer 124 is retained. Clip 126 preferably includes an opening 126a for receiving a free end portion of transducer 124 as shown for example in FIG. 5B. Shoe 122 also may be provided with a silicone window 130 to serve as a buffer between a patient's skin and transducer 124. Silicone is transparent to ultrasound and thus may be used to assist in creating a sterile field. The silicone window for example may be disposable, as may be shoe 122.

Shoe 122 may be mounted on a base 132 for linear translation thereon. In particular, as shown in FIG. 5D, shoe 122 may include a boss 122c which is threadably received on a lead screw 134 and disposed in a slot 132a in base 132. In particular, boss 122c is sized to fit in slot 132a such that shoe 122 is only permitted to travel linearly along the axis 135 defined by lead screw 134 but not rotate thereabout. With further reference to FIGS. 3E-3F, lead screw 134 has a collar 136 thereon proximate a free end thereof. Collar 136 abuts base 132 and is releasably fixed to a smooth portion 134a of lead screw 134 by a set screw 138. A bushing 140 is provided proximate an opposite end of lead screw 134 and is disposed in a hole in base 132 and bears against a shoulder portion therein. A knob 142 is provided to turn lead screw, which is captive in slot 132a.

Thus, boss 122c travels along axis 135 when knob 142 is turned, but travel of boss 122c and thus shoe 122 is limited by front and rear walls 132b, 132c.

As shown in FIG. 5G, a variety of sizes of transducers may be accommodated such as transducer 124b.

Finally, a coupling portion 144 may be provided as described with respect to previous embodiments, so that device 120 may be coupled to a curvilinear articulating arm.

In use, ultrasound transducer 124, 124b and optionally silicone window 130 may serve as a compression region that may directly contact a patient proximate the anatomical region of interest for vascular tamponade. Coupling portion 144 of device 120 initially may be secured to an articulating arm assembly as will be described, which for example may be mounted to the railing of a hospital bed or otherwise fixed to a stable object. The operator of device 120 may first position transducer 96 secured in shoe 122 proximate the anatomical region of interest on the patient's skin over an entry wound or hole in a vessel and apply a desired amount of pressure. With a second hand, the operator may then couple device 122 to an articulating arm. Once transducer 124, 124b is held in a desired orientation and position to provide a desired pressure on the hole in the vessel, the articulating arm may be locked in position as will be further described so that transducer 124, 124b remains in place as set by the operator to provide generally constant pressure for a desired period of time. The puncture and healing thereof may be monitored by an operator using ultrasound images from transducer 124, 124b.

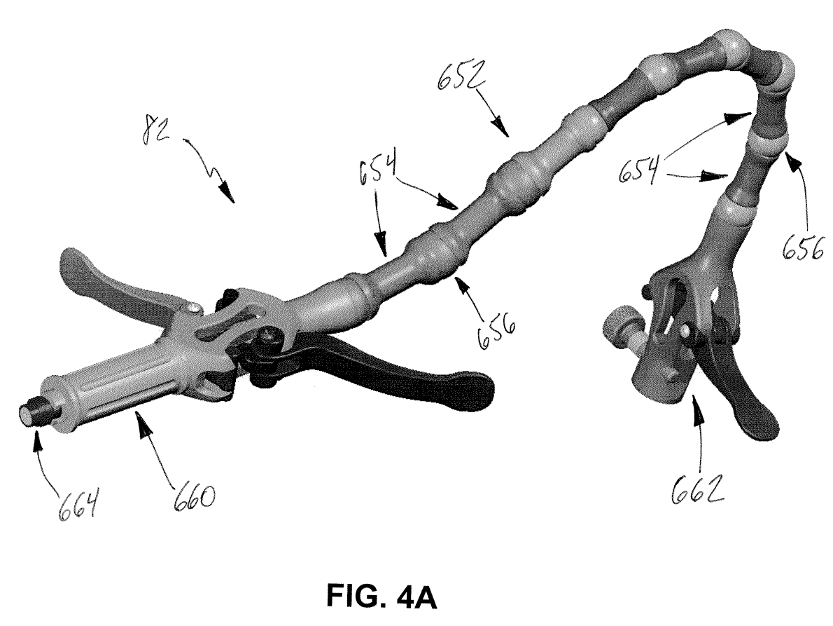

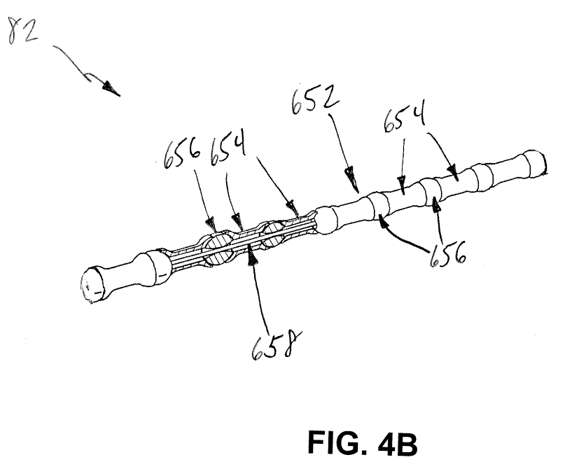

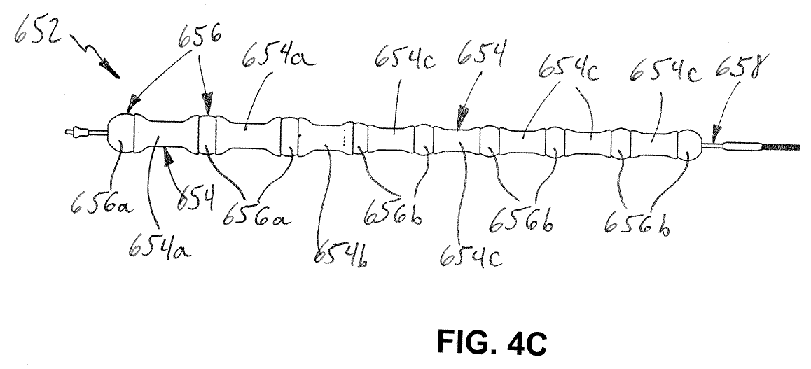

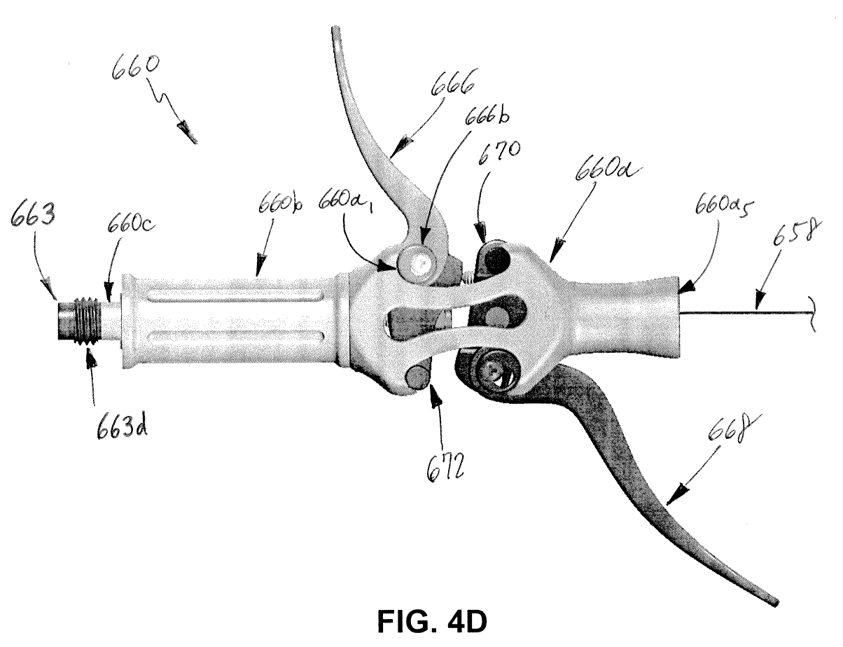

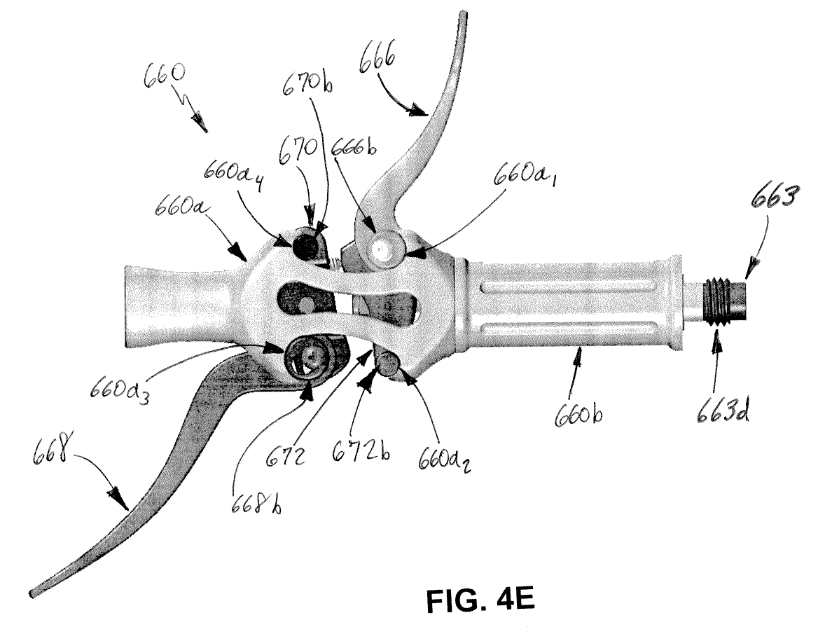

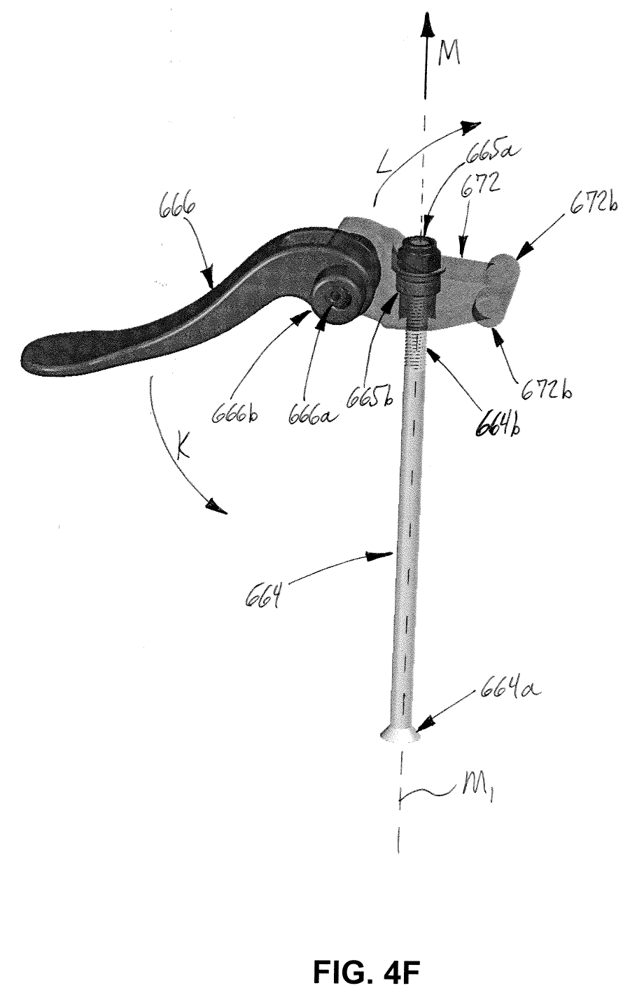

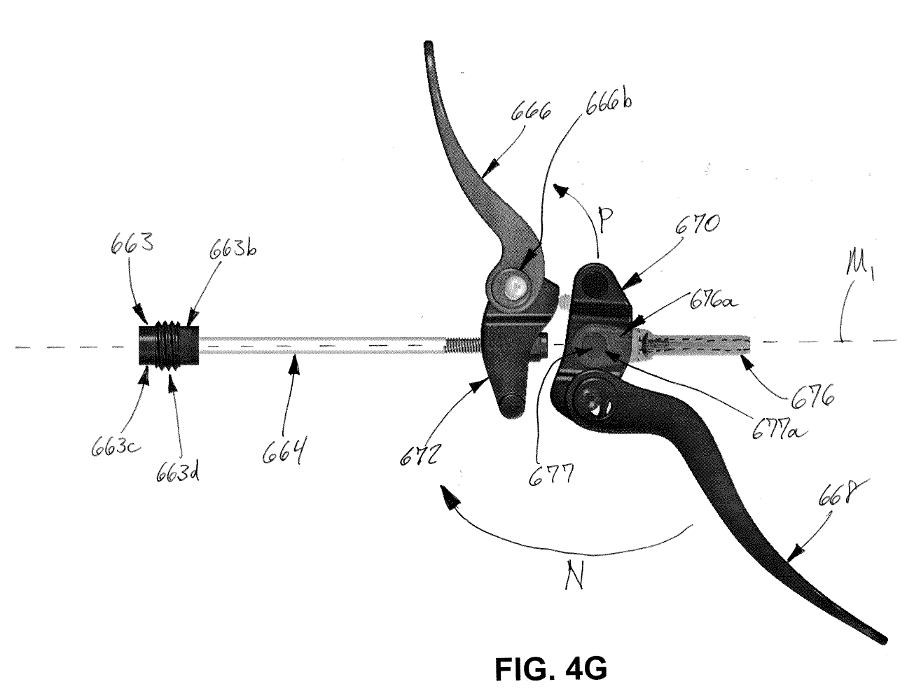

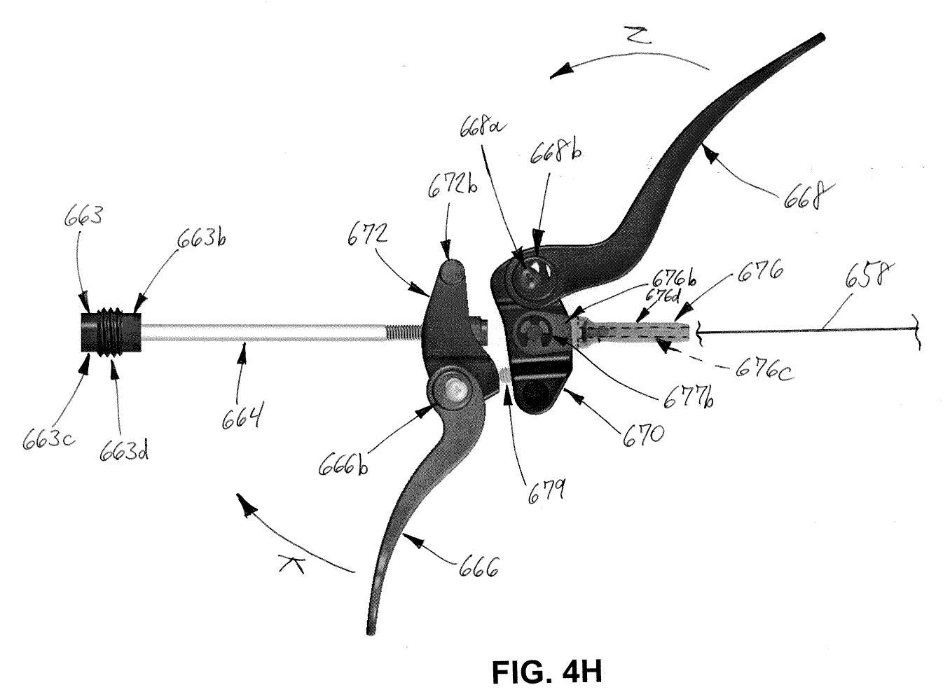

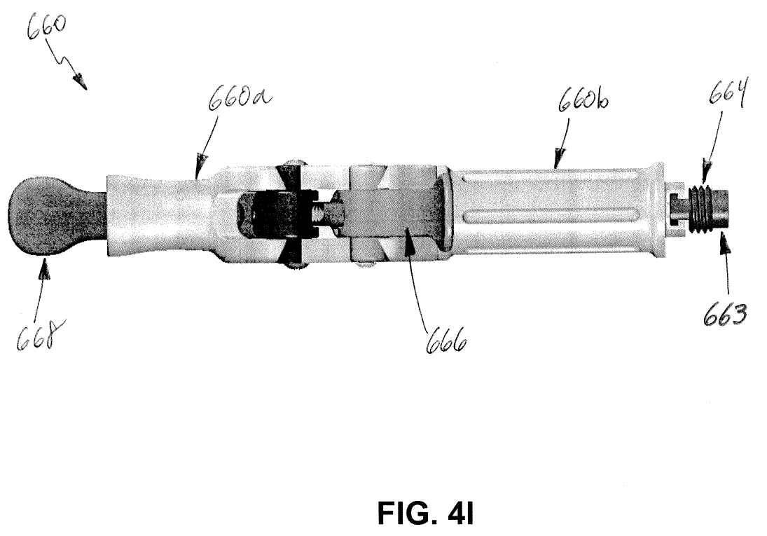

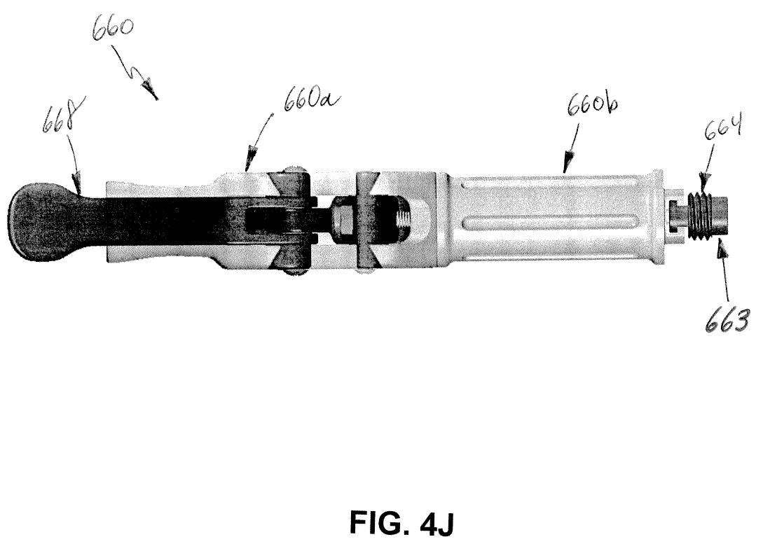

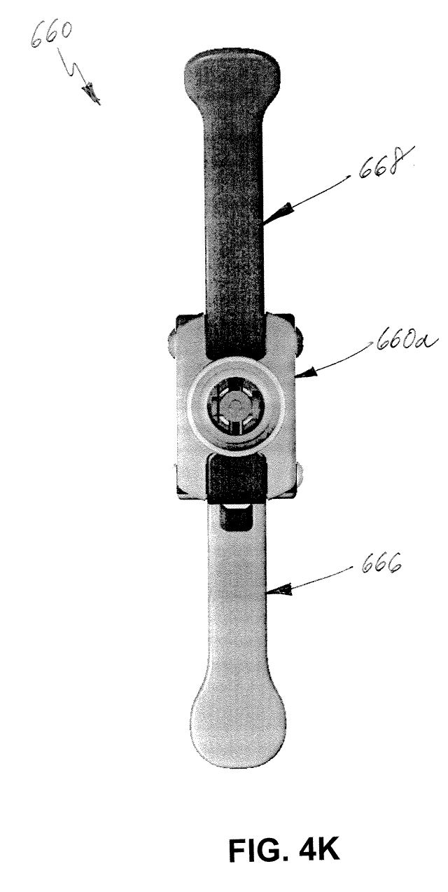

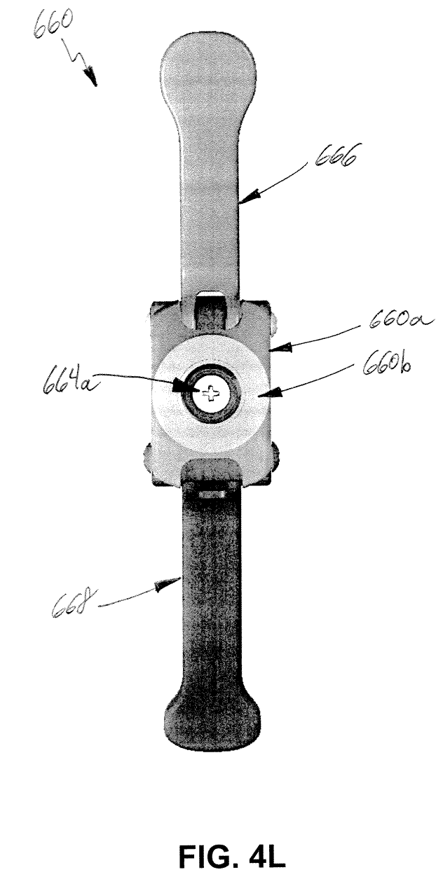

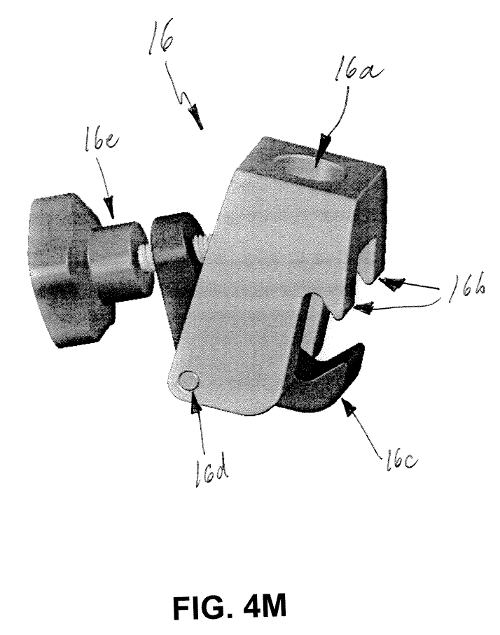

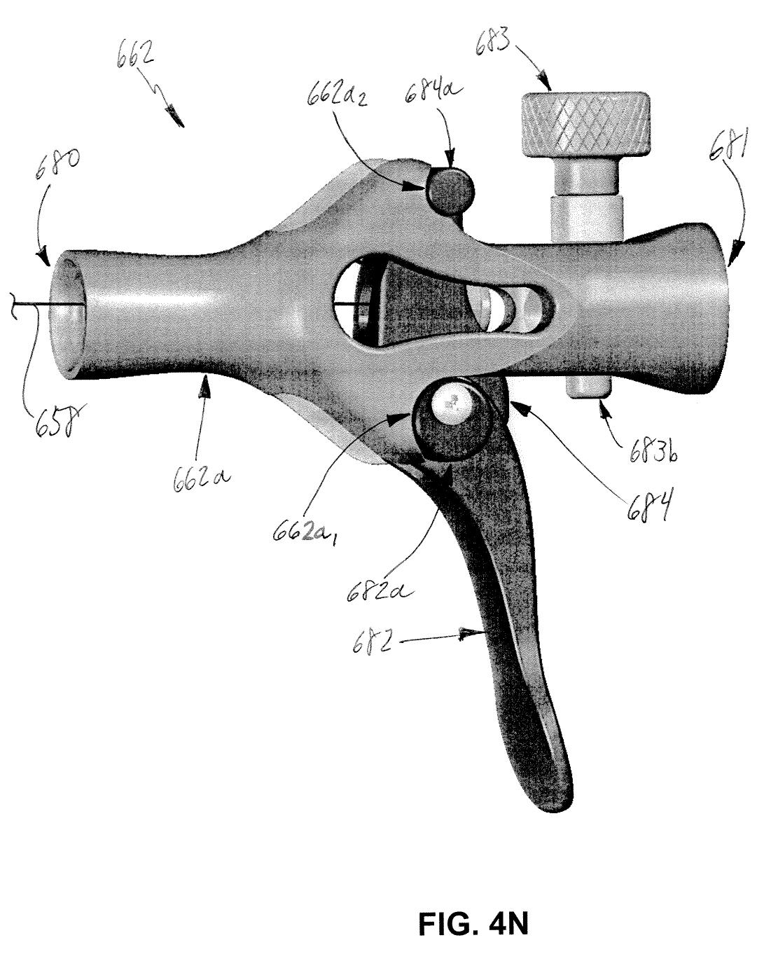

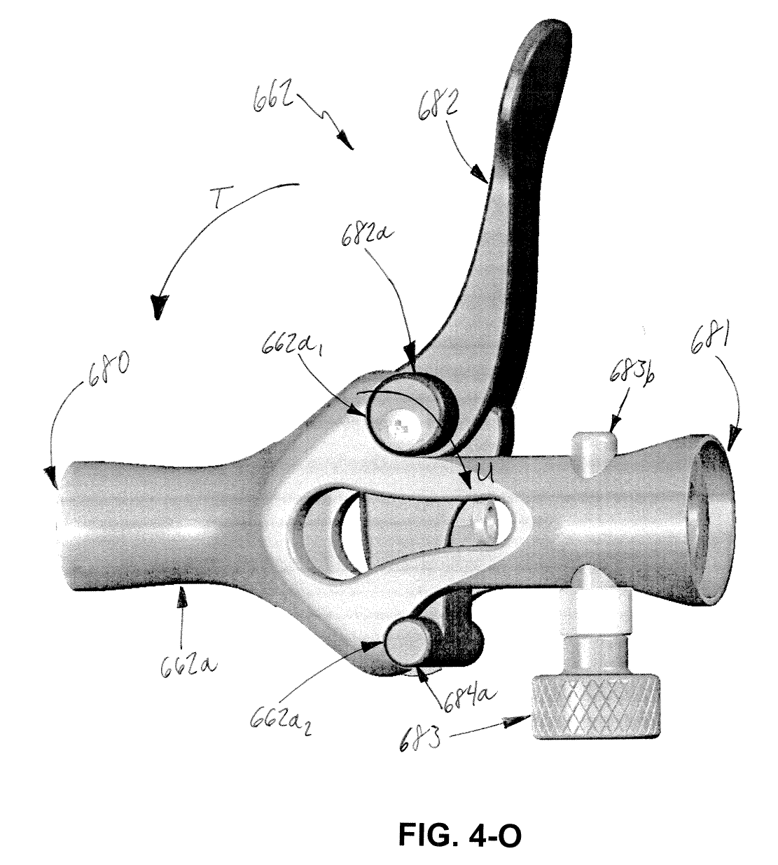

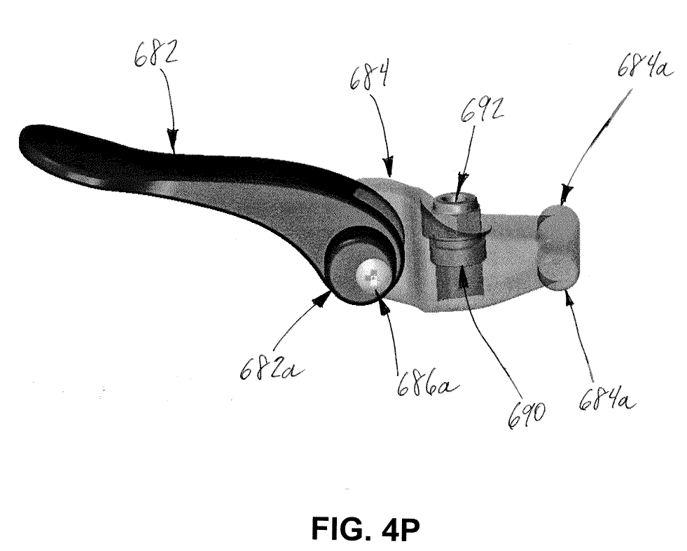

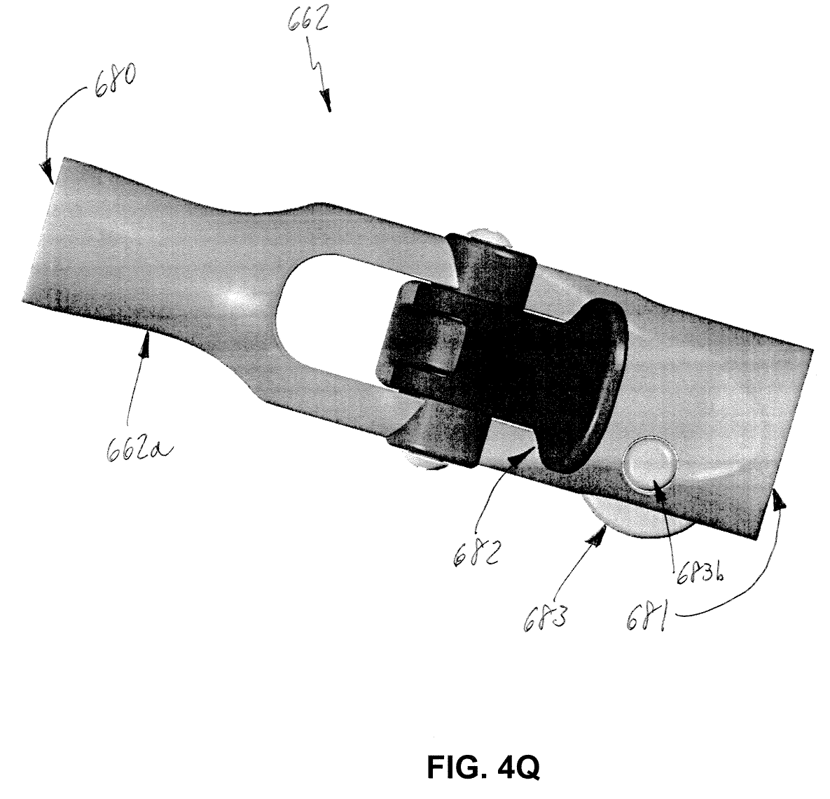

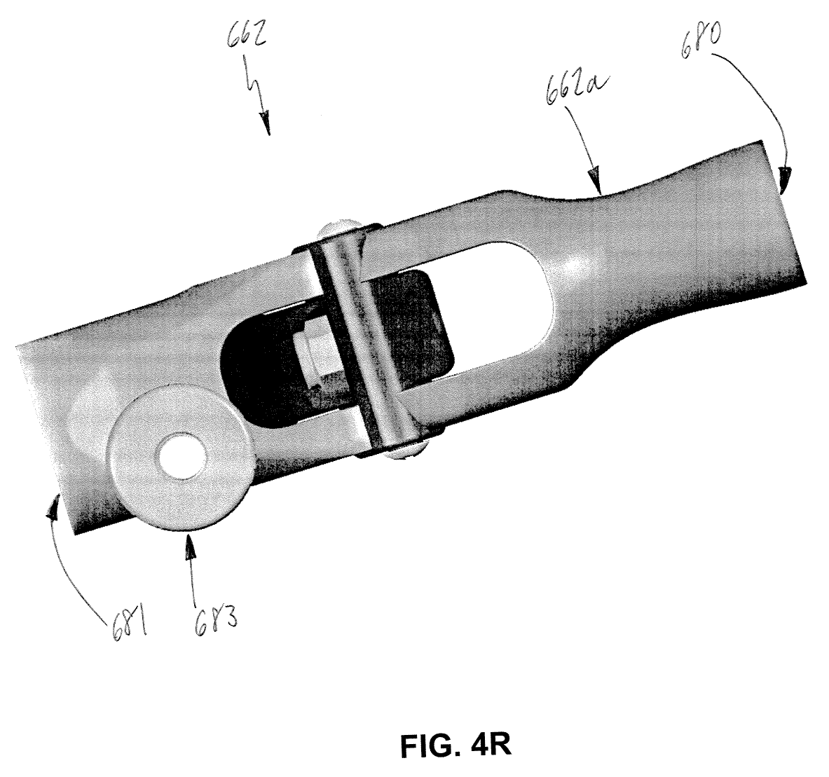

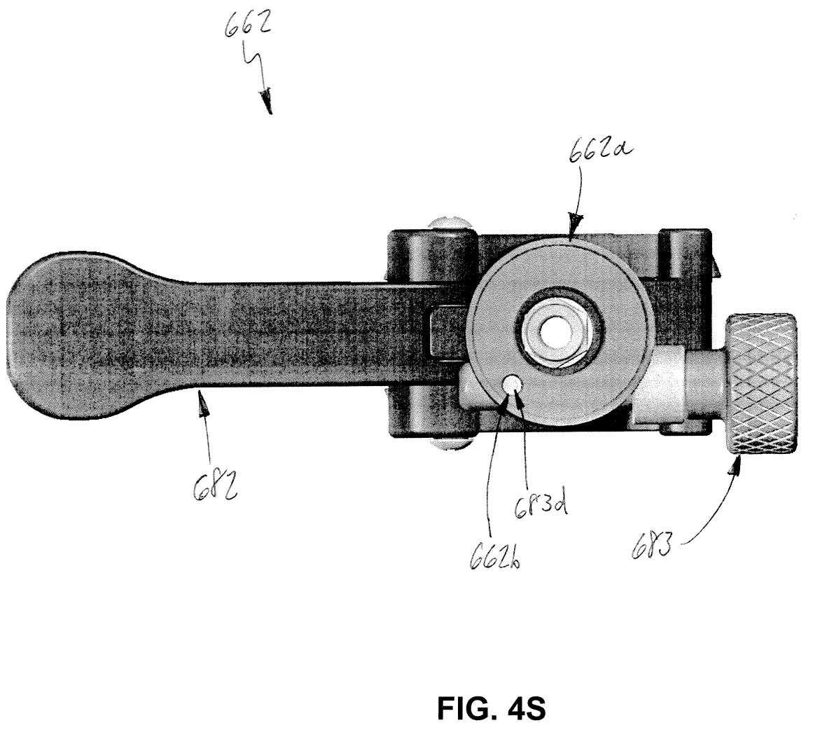

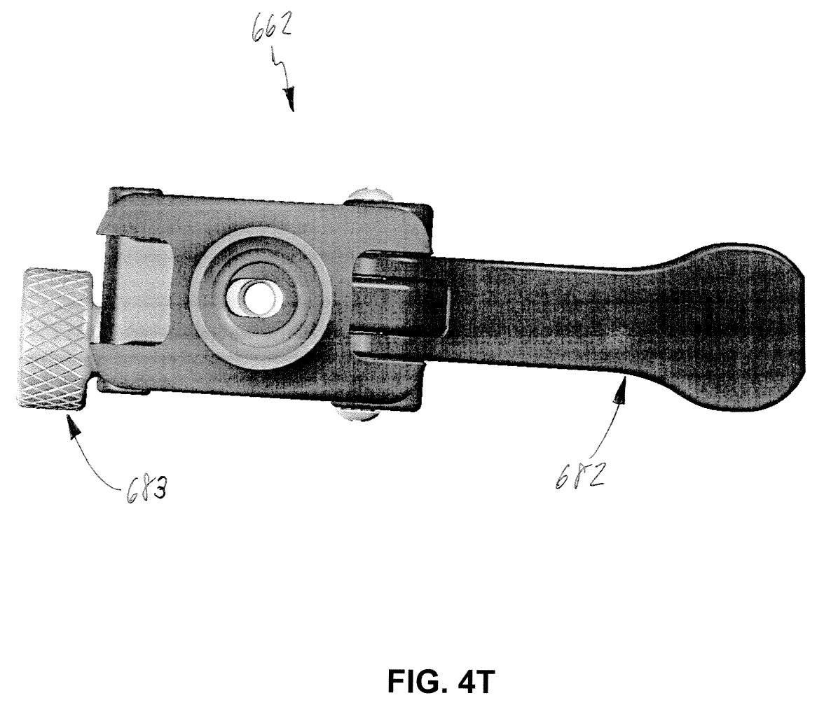

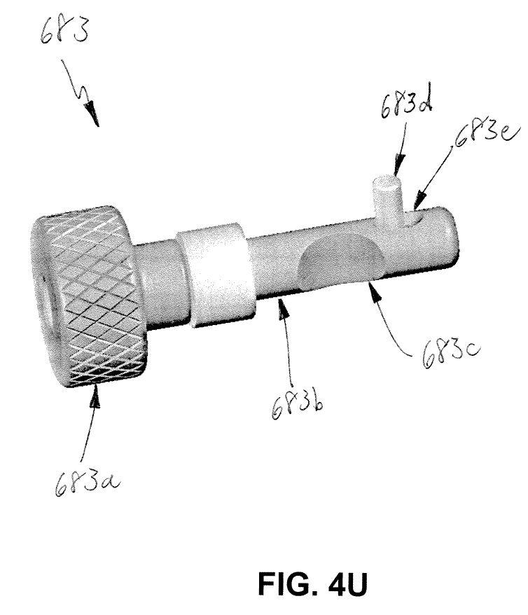

Turning to FIGS. 4A-4C, an exemplary preferred curvilinear articulating arm assembly 82 is shown for use with a device 10, 20, 40, 90, 120. Arm assembly 82 includes a central arm 652 with a ball-sleeve arrangement that forms joints. In particular, central arm 652 includes a plurality of sleeves 654 with spherical balls 656 disposed therebetween thus forming ball and socket connections. In the exemplary embodiment shown in the figures, three balls 656a of a first size are disposed adjacent one another proximate one end of arm 652, while the remaining balls 656b are of a second size smaller than the first size. Sleeves 654a of a first size and sleeves 654c of a second size smaller than the first size are provided for accommodating balls 656a, 656b, respectively, while a transition sleeve 654b is provided intermediate sleeves 654a, 654c as shown for accommodating a ball 656a on one side and a ball 656b on the other side thereof. Sleeves 654 are configured and dimensioned to receive balls 656a, 656b at ends thereof and thus permit articulating of sleeves with respect to each other. A tensioning wire 658 runs generally centrally through sleeves 654 and balls 656, as will be further described shortly.

Preferably, wire 658 is formed of metal. In an exemplary preferred embodiment, wire 658 is Type 302 stainless steel wire rope, 1×19 strand, 5/32 inch diameter, with a breaking strength of 3300 lb. (McMaster-Carr part number 3458T27). One exemplary operation of a wire tensioning mechanism is shown and described in U.S. Pat. No. 3,858,578 to Milo, which is expressly incorporated herein by reference thereto. Preferably, curvilinear articulating arm assembly 82 may move with six degrees of freedom.

In the exemplary preferred embodiment, three additional balls 656a and three additional sleeves 654a are provided to the arm assembly 82 shown in FIGS. 4A-4C, with arm assembly 82 having a fully extended (straightened) length of about 40 inches. In other embodiments, other desired lengths of arm assembly 82 may be accomplished by changing the number of balls and sleeves. For example, without the three additional balls 656a and three additional sleeves 654a, arm assembly 82 may have a length of about 32 inches.

A base handle 660 is coupled to central arm 652 on a first end thereof, preferably adjacent a ball 656a. In addition, a free handle 662 is coupled to central arm 652 on a second end thereof, preferably adjacent a ball 656b.

In one preferred exemplary embodiment, a series of larger balls 656a is provided proximate base handle 660 to provide stability to curvilinear articulating arm assembly 82. If for example a user such as a surgeon orients assembly 82 by grasping it proximate free handle 662, substantial bending forces may be exerted on central arm 652 proximate base handle 660. Thus, the use of larger balls 656a proximate base handle 660 as compared to smaller balls 656b proximate free handle 662 provides a system with larger surface area balls near base handle 660 for additional resistance to rotational movement in that portion of central arm 652 and thus more stability. In alternate embodiments, more than two different sizes of balls 656 or more than two sets of sizes of balls 656 may be used, preferably increasing in size toward base handle 660. In one alternate embodiment, each of the balls 656 in central arm 652 is of increasingly larger size from free handle 662 to base handle 660. The use of only two sizes of balls 656 advantageously facilitates manufacture and construction of arm assembly 82 because of the need to only stock two sizes as compared to a larger number of sizes and concomitantly greater case of construction because only two sizes need be assembled to form central arm 652. In yet another alternate embodiment, central arm 652 may be formed of balls 656 that all are the same size.

Turning to FIG. 4D-4L, base handle 660 will be described. Base handle 660 includes a body portion 660a with levers 666, 668 pivotably associated therewith, as well as an extension 660b that turns screw coupling 663 and rotates in relation to and independent of body portion 660a. Base handle 660 further includes cam mechanisms 670, 672 as will be described. Portion 663b of coupling 663 preferably is noncircular and mechanically engages and is fixed to a like-shaped and sized non-circular opening in portion 660c of extension 660b so that rotation of extensions 660b as by gripping and turning by a user imparts like-rotation of coupling 663 for example for demountable coupling to clamp 16 and further coupling to a surgical table rail 18, as shown for example in FIG. 1. In the preferred exemplary embodiment, coupling 663 comprises a threaded portion 663d which may be threadably received in a threaded hole 16a disposed in clamp 16.

Coupling 663 is disposed proximate a first free end 664a of a stainless steel shaft 664 which extends therethrough and is provided with a head that abuts a shoulder disposed in end 663c of coupling 663. Preferably, rotation of coupling 663 is independent of rotation of shaft 664. Shaft 664 preferably extends through a hole in extension 660b.

Lever 666 is pivotably coupled to rocker arm 672 with a pin 666a that is disposed such that rotation of lever 666 results in eccentric movement of rocker arm 672. As shown for example in FIGS. 4D-4E, cylindrical projections 666b of lever 666 are received and rotate in arcuate cradle portions 660a₁ of body portion 660a, while cylindrical projections 672b of rocker arm 672 are received and rotate in arcuate cradle portions 660a₂ of body portion 660a. Rotation of lever 666 toward screw coupling 663 in direction K lifts pin 666a, and because rocker arm 672 rests on pin 666a, rocker arm 672 is rotated in direction L in an eccentric fashion.

As seen particularly in FIG. 4F, shaft 664 includes a threaded portion 664b the free end of which is threadably associated with a nut 665a. Shaft 664 extends through a hole in rocker arm 672 and an unthreaded insert 665b with a hole therein which assists in guiding travel of rod 664 along the longitudinal axis thereof. Pivoting of lever 666 in direction K causes rotation of rocker arm 672, and with shaft 664 coupled to nut 665a and nut 665a abutting insert 665b, rod 664 is translated in direction M.

When coupling 663 is threaded into a like threaded hole by rotation of extension 660b, arm assembly 82 is relatively loosely coupled by the connection of coupling 663 to the hole. To firmly couple arm assembly 82, lever 666 may be pivoted in direction K so that threaded portion 663d of coupling 663 also moves in direction M and bears against the threads of the hole in which it is received. The leverage created by even slight movement of the threads against the threaded holes, on the order of tens of thousandths of an inch, creates a wedging effect that strongly locks arm assembly 82 to the hole.

Lever 668 of base handle 660 also is pivotably coupled to a rocker arm 670 with a pin 668a that is disposed such that rotation of lever 668 results in eccentric movement of rocker arm 670. As shown for example in FIGS. 4D-4H, cylindrical projections 668b of lever 668 are received and rotate in arcuate cradle portions 660a₃ of body portion 660a, while cylindrical projections 670b of rocker arm 670 are received and rotate in arcuate cradle portions 660a₄ of body portion 660a. Rotation of lever 668 toward screw coupling 663 in direction N lifts pin 668a, and because rocker arm 670 rests on pin 668a, rocker arm 670 is rotated in direction P in an eccentric fashion.

A forked member 676, which for example may be formed of stainless steel, is coupled to rocker 670 and includes substantially parallel prongs 676a, 676b which mate with side walls of rocker 670 as shown. Rocker 670 is pivotably associated with forked member 676, with a shaft 677 extending through aligned holes in prongs 676a, 676b and rocker 670. Shaft 677 may be provided with a head 677a and an external retaining ring 677b secured in a shaft groove proximate an end opposite head 677a to retain forked member 676 in association therewith and thus with rocker 670. An axial through hole 676c is provided in tubular portion 676d of forked member 676. Tensioning wire 658 is coupled to forked member 676 by inserting an end portion of wire 658 in hole 676c and swaging tubular portion 676d so that wire 658, which extends out of open end 660a₅ of body portion 660a, is retained by compression within tubular portion 676d.

When lever 668 is rotated in direction N, shaft 676 translates along the longitudinal axis M₁ toward coupling 663 creating substantial tension in tensioning wire 658 such that movement of curvilinear articulating arm assembly 82 may be substantially resisted. In particular, actuation of second lever 668 may increase or decrease the tension in wire 658 as desired by acting on rocker arm 670. By increasing tension in wire 658, central arm 652 preferably becomes increasingly resistant to movement although central arm 652 preferably still may be moved through its full range of motion. Thus, a user may orient curvilinear articulating arm assembly 82 as desired, and then increase the tension of wire 658 so that the orientation of arm 652 is releasably fixed. Lever 668 preferably has an angular range of movement about pin 668a of up to about 180° to permit substantial tension to be generated in tensioning wire 658.

Rockers 670, 672 preferably are associated with each other as with a spring plunger 679 extending from within one rocker 670 into a hole in the other rocker 672. Spring plunger for example may be a stainless steel spring plunger with a round Delrin nose, without a lock element, with ¼″-20 threading, and 3-13 lb. end force (McMaster-Carr part number 84765A33). Spring plunger 679 is used as shown because under the force of gravity, first lever 666 may otherwise tend to move toward a closed position with in the direction of arrow K. Instead, spring plunger 679 applies pressure to rocker arm 672 to set lever 666 to tend to a default open position in which shaft 664 has not otherwise been raised toward open end 660a₅ of body portion 660a.

In a preferred exemplary embodiment, rocker 670 moves with substantially greater eccentricity than rocker 672.

Clamp 16 for use with base handle 660 may be demountably attached to surgical table rail 18. As previously discussed, actuation of first lever 666 permits a user to apply a force on coupling 663 so that movement is resisted (e.g., in response to an 8 or 10 pound force applied to arm 652). In an alternate embodiment which will be further described later, screw coupling 664 as shown in FIG. 4A proximate base handle 660 of arm assembly 82 may be threadably associated with a threaded hole in another support surface.

A preferred exemplary embodiment of clamp 16 is shown in FIG. 4M. Clamp 16 includes a threaded hole 16a for threadably receiving threaded portion 664 of base handle 660. In addition, clamp 16 includes fixed jaw portion 16b and movable jaw portion 16c which is pivotable about axle 16d and lockable in place using screw mechanism 16e to firmly couple clamp 16 to a rail 18 secured between jaw portions 16b, 16c.

Next turning to FIGS. 4N-4U, free handle 662 will be described. Free handle 662 includes a wire receiving portion 680 and an end effector receiving portion 681. In particular, wire receiving portion 680 preferably is configured to receive a ball 656h therein, along with an end portion of wire 658. As described previously with respect to base handle 660, a pivotable lever 682 is associated with free handle 662 and preferably is coupled to tensioning wire 658 so that actuation of lever 682 may increase or decrease the tension in wire 658 as desired by acting on rocker arm 684. By increasing tension in wire 658, central arm 652 preferably becomes less flexible. Thus, a user may orient curvilinear articulating arm assembly 82 as desired, and then increase the tension of wire 658 so that the orientation of arm 652 is releasably fixed. Free handle 662 has a body portion 662a, and lever 682 is rotatable with respect thereto. An interface lock 683 also is rotatably associated with body portion 662a proximate end effector receiving portion 681, as will be described shortly.

Lever 682 is pivotably coupled to rocker arm 684 with a pin 686a that is disposed such that rotation of lever 682 results in eccentric movement of rocker arm 684. Cylindrical projections 682a of lever 682 are received and rotate in arcuate cradle portions 662a₁ of body portion 662a, while cylindrical projections 684a of rocker arm 684 are received and rotate in arcuate cradle portions 662a₂ of body portion 662a. Rotation of lever 682 toward wire receiving portion 680 in direction T lifts pin 686a, and because rocker arm 684 rests on pin 686a, rocker arm 684 is rotated in direction U in an eccentric fashion.

Rocker arm 684 includes a hole in which a self-aligning setup washer 690 (a two-piece washer with one portion that rocks in another portion) is disposed. Setup washer 690 for example may be an 18-8 stainless steel self-aligning setup washer, 4 inch in size, 17/64 inch inner diameter, ±2 inch outer diameter, and 0.250 inch to 0.281 inch thick (McMaster-Carr part number 91944A028). A nut 692 also may abut setup washer 690 on the flat upper surface thereof and rock thereon. A threaded stud (not shown) may be swaged to the end of tensioning wire 658 opposite the end attached to forked member 676, thus coupling wire 658 to the threaded stud by compression. The threaded stud may in turn be threadably associated with nut 692. Wire 658 is provided with suitable length to span from forked member 676 to nut 692.

Pivoting of lever 682 in direction T causes rotation of rocker arm 684, and with tensioning wire 658 coupled to nut 692 and nut 692 abutting insert 690, tension in wire 658 may be increased. In particular, actuation of lever 682 may increase or decrease the tension in wire 658 as desired. By increasing tension in wire 658, central arm 652 preferably becomes increasingly resistant to movement although central arm 652 preferably still may be moved through its full range of motion. Thus, a user may orient curvilinear articulating arm assembly 82 as desired, and then increase the tension of wire 658 so that the orientation of arm 652 is releasably fixed. Lever 668 preferably has an angular range of movement about pin 686a of up to about 90° to permit tension to be generated in tensioning wire 658.

In the preferred exemplary embodiment, actuation of lever 682 free handle 662 permits initial tensioning of central arm 652 while still permitting restricted movement. And, actuation of lever 668 of base handle 660 permits substantially greater tensioning of central arm 652 while also still permitting restricted movement thereof.

Advantageously, with tension created in wire 658 of central arm 652 to restrict movement thereof, the orientation of lever 668 such as with respect to a patient still may readily be reset or adjusted before lever 666 in base handle 660 is actuated to create sufficient force to prevent rotation of threaded portion 663d of coupling 663 in the hole in which it is received.

As shown in FIG. 4U, interface lock 683 includes a knurled knob portion 683a and a cylindrical post 683b that is provided with an arcuate cutout 683e. Interface lock 683 is coupled to body portion 662a with set screw 683d which is threadably received in a threaded hole 662b in body portion 662a. Set screw 683d is further received in a slot 683e in post 683b to lock post 683b in a position with arcuate cutout 683c oriented to be movable along the longitudinal axis of cylindrical post 683b.

Cylindrical post 683b may be disposed in a disengaged position in which the axial position of post 683b is such that arcuate cutout 683c generally follows the inner cylindrical contour of end effector receiving portion 681. Also, cylindrical post 683b may be disposed in an engaged position in which the axial position of post 683b is such that a portion of cylindrical post 683b other than arcuate cutout 683c extends past the inner cylindrical contour of end effector receiving portion 681 toward the central longitudinal axis of end effector receiving portion 681.

In use, in order for example to couple articulating arm assembly 82 to an end effector such as a device 90, by capturing post 94aa₁ of coupling portion 94aa of device 90 in end effector receiving portion 681 of free handle 662, post 94aa₁ is inserted therein while interface lock 683 is disposed in the aforementioned disengaged position.

While lock 683 is in the disengaged position, post 94aa₁ may freely rotate about the central axis of receiving portion 681. Once a desired orientation is set, lock 683 may be translated along the major axis defined by slot 683a so that a portion of cylindrical post 683b of lock 683 is disposed in an engaged position and bears against post 94aa₁. Such interference between post 94aa₁ of device 90 and post 683b of lock 683 provides sufficient pressure so that post 94aa₁ will remain fixed in rotational position and translation along the longitudinal axis thereof against the inner cylindrical contour of end effector receiving portion 681.

In one method of conducting vascular tamponade according to the present invention, a curvilinear articulating arm assembly 82 with base attachment 73 is releasably secured to a surgical table rail 72. Device 90 is demountably coupled to the free end of arm assembly 82 at free handle 662. By articulating the lever 682 at the free end to a locked position, arm assembly 82 will hold position when left alone but can be easily repositioned with one hand without having to loosen or unclamp any other mechanisms. In this mode, arm assembly 82 should have sufficient resistance to hold device 90 in position absent other external forces, much like a gooseneck lamp. If locking lever 668 near the base of arm assembly 82 is also locked then arm assembly 82 will hold position against a much greater force, but this lever 668 will then have to be released when ready movement of the arm/device combination is required.

Once curvilinear articulating arm assembly 82 is fixed in position, curvilinear articulating arm assembly 82 is designed to result in reliable position holding for device 90. There is no need to adjust any locking or tensioning mechanisms because of the geometry of the setup and the resistance provided by the arm in its “gooseneck lamp” mode.

In some methods, gross movements of the device—for example to move the device away from a puncture that has had sufficient clotting and thus no longer needs pressure to be applied—may be accomplished by grabbing the articulating arm assembly 82 proximate the swivel joints and reorienting the device from that gripping point. For smaller movements, it is possible to simply grab and torque the device 90 itself.

Each of the devices 10, 20, 40, 90, 120 described herein may be used in accordance with the aforementioned methods, and each of the examples above with respect to device 90 also may be applied to devices 10, 20, 40, 120.

Also, although an exemplary curvilinear articulating arm assembly is described herein, it should be understood that other, preferably curvilinear articulating arm assemblies instead may be used which preferably provide six degrees of freedom of movement and permit relatively rigid positioning such as described herein.

In some embodiments, other retaining systems may be employed to position a device 10, 20, 40, 90, 120 in fixed relationship to a puncture without the need for continuing to manually apply pressure. Such retaining systems preferably offer multiple degrees of freedom.

In some embodiments of the present invention, a device for vascular tamponade such as an arm 82 and device 40 as shown in FIG. 1N may be coupled to a patient support other than a rail of a table. For example, referring next to FIG. 5, an exemplary support system 710 according to the present invention is shown with a variety of components coupled thereto. Support system 710 includes a tray 712, curvilinear articulating arm assemblies 82, 716 having respective end effectors 100, 720, an IV pole 722, an arm board 724, and rail assemblies 726, 728. A variety of end effectors may be demountably attached for example to articulating arm assembly 716 to assist a technician or practitioner with a medical/imaging procedure or provide other features useful with respect to a patient. End effector 720, for example, is configured as a self-centering abdominal probe bracket.

In one preferred exemplary embodiment, tray 712 may include two pairs of hold regions 730, each pair being disposed proximate a free cranial end 732 or free caudal end 734 of tray 712. In alternate embodiments, other numbers of hold regions 730 may be provided such as two or more, and hold regions 730 may be provided in other regions of tray 712 such as intermediate ends 732, 734 proximate sides 736, 738. Hold regions 730 may be configured as hand holds, or alternatively may be configured to receive strapping so that tray 712 may be releasably coupled to another object such as an ambulance stretcher, hospital bed, operating room table, or imaging scanner table. In some embodiments, handles may be coupled to tray 712. As also shown in FIG. 5, attachment regions 740 are provided proximate sides 736, 738 for demountably coupling components as previously described to tray 712, as will be farther described below. In the exemplary preferred embodiment, tray 712 is provided with thirteen attachment regions 740, although in alternate embodiments another number of regions 740 may be provided such as at least one or tray 712 may be provided with a surgical rail or track permitting substantial freedom of coupling of components along the length thereof.

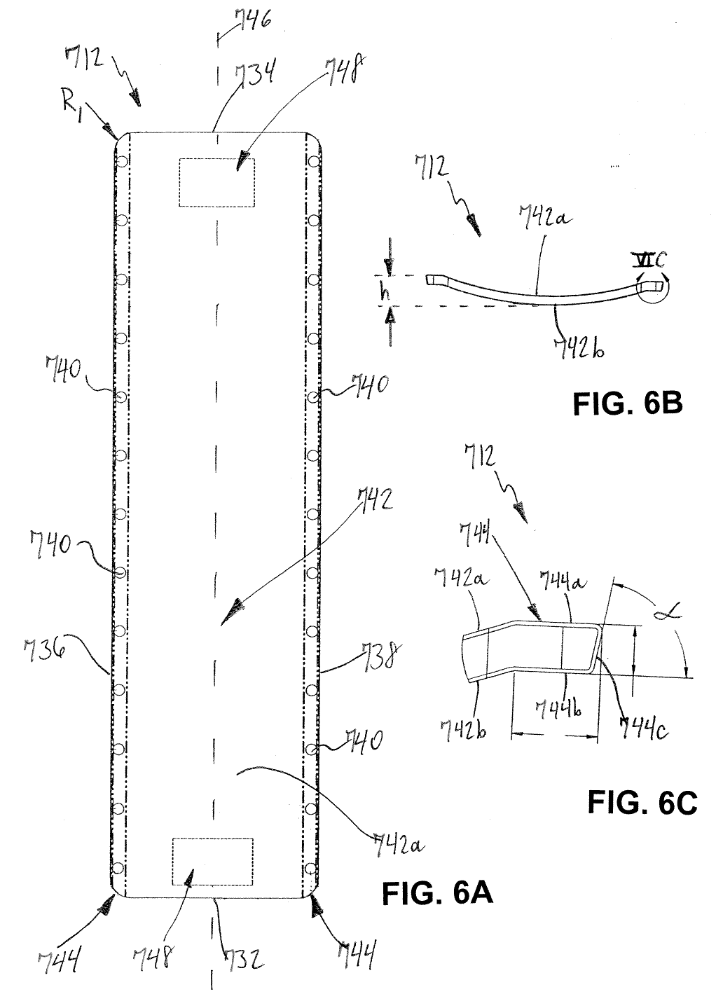

Turning to FIGS. 6A-6C, additional features of tray 712 are shown. Although hand hold regions 730 are not included in the figure, such regions may be provided as shown in FIG. 5. Attachment regions 740 are provided in spaced arrangement along the perimeter of tray 712. Preferably, tray 712 includes a central arcuate portion 742 disposed between outer ledge portions 744. Preferably, regions 740 are provided on outer ledge portions 744. Central arcuate portion 742 preferably has an upper concave surface 742a for receiving a patient and optionally a cushion (not shown) for the patient to rest against, and optionally includes a lower convex surface 742b. Preferably, outer ledge portions 744 include upper and lower surfaces 744a, 744b connected by a sidewall 744c at an angle α with respect to surface 744b. In a preferred exemplary embodiment, sidewall 744c is disposed at an angle α between about 60° and about 100°, more preferably between about 70° and about 90°, and most preferably at about 80°.

In a preferred exemplary embodiment, tray 712 is formed of natural finish carbon fiber, R-51 foam core, and phenolic. Attenuation preferably is less than 1 mm Al equivalency. Thus, tray 712 is radiolucent and suitable for use with computed axial tomography (CT) scanners. In other embodiments, tray 712 is formed of a material suitable for use with magnetic resonance imaging (MR) scanners. In addition, tray 712 preferably supports a load of 900 lbs. evenly distributed along centerline 746, about which tray 712 may be substantially symmetric as shown. Indicia 748 optionally may be provided, as shown for example proximate ends 732, 734. The indicia may for example indicate preferred orientation of tray 712 with respect to a patient lying thereon.

In the preferred exemplary embodiment, attachment regions 740 on each side of tray 712 are evenly spaced from each other by about 6 inches between centers thereof. To accommodate patients and equipment attached to tray 712, in one preferred embodiment tray 712 has a length of about 78 inches, a width of about 21 inches, a generally uniform thickness of about 0.9 inch, and a height h of about 2.5 inches.

Corners may be provided with a radius R₁ of about 2 inches. In the preferred exemplary embodiment, attachment regions 740 preferably accommodate threaded inserts, which may be formed of aluminum.

In some embodiments, tray 712 is sized to hold an adult patient, and may be between about 180 cm and about 200 cm long. However, it will be appreciated that longer and shorter trays may be provided. In order to accommodate an adult patient, tray 712 may support an overall weight capacity of at least about 200 pounds, and preferably at least about 300 pounds. However, if a tray 712 is sized for use with a pediatric patient, tray 712 may only accommodate weights that do not exceed 200 pounds, and more preferably do not exceed 100 pounds.

Although the surface of portion 742 of tray 712 is substantially smooth in the preferred exemplary embodiments in alternate embodiments the surface may be textured to provide additional resistance to motion of objects and/or a patient placed thereon.

Tray 712 thus is suitable for use in multiple environments, and thus may “move” with the patient from one environment (e.g., ambulance) to the next (e.g., CT scanner) without removing a patient supported thereon.

While various descriptions of the present invention are described above, it should be understood that the various features can be used singly or in any combination thereof. Therefore, this invention is not to be limited to only the specifically preferred embodiments depicted herein. Further, it should be understood that variations and modifications within the spirit and scope of the invention may occur to those skilled in the art to which the invention pertains. For example, each of devices 10, 20, 40, 90, 120 described herein may be used with an articulating arm that may be mounted to the railing of a hospital bed or otherwise fixed to a stable object. Also, each of devices 10, 20, 40, 90, 120 may be fixed to stable objects without the use of an articulating arm as described herein; for example, a vise or simple clamp may be used to grasp the coupling portion of each of the devices preferably with the vise or clamp being in a fixed position with respect to the patient. Moreover, although the use of ultrasound transducer for providing pressure proximate punctures has been disclosed herein, other imaging devices are contemplated to be used instead of ultrasound transducers. For example, devices for detecting hematoma may be secured in brackets 92, 122 and positioned to apply constant pressure proximate a puncture. A variety of imaging devices may be secured in brackets 92, 122 such as infrared thermal imaging systems. Even further, the devices and methods disclosed herein for vascular tamponade may be used in combination with scanning technologies such as magnetic resonance imaging (MR) or computed axial tomography (CT). Based on results of such imaging, pressure applied by a device 10, 20, 40, 90, 120 proximate the puncture may be varied or adjusted for example as a result of identification of a hematoma.

Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A device for vascular tamponade comprising:
a compression portion;
a curvilinear articulating arm coupled to the compression portion.

2. The device of claim 1, wherein the compression portion is demountably coupled to a body portion.

3. The device of claim 2, wherein the compression portion is slidably associated with the body portion.

4. The device of claim 1, wherein the compression portion is spring-loaded.

5. The device of claim 1, wherein the compression portion comprises a first portion with a through hole therein and a second portion for mating with the hole.

6. The device of claim 1 wherein the compression portion comprises an ultrasound transducer.

7. The device of claim 6, further comprising a bracket, wherein the ultrasound transducer is retained in the bracket.

8. The device of claim 1, wherein the bracket is coupled to a base portion and linearly moveable with respect thereto.

9. The device of claim 5, wherein the base portion comprises a linear screw and the bracket comprises a boss, the linear screw being threadably associated with the boss.

10. A device for vascular tamponade comprising:
a bracket;
a transducer coupled to the bracket;
a curvilinear articulating arm coupled to the bracket.

11. The device of claim 10, wherein the bracket comprises a silicone window disposed proximate an imaging region of the transducer.

12. The device of claim 10, wherein the bracket comprises a recessed portion for receiving an imaging portion of the transducer and a clamping portion for securing a different portion of the transducer.

13. The device of claim 10, wherein the bracket is coupled to a base portion and constrained to linear movement with respect thereto.

14. A method for vascular tamponade comprising:
manually applying a desired pressure proximate a puncture in a vessel by positioning a compression member to apply pressure against skin of a patient, the desired pressure permitting clot formation at the puncture;
coupling the compression member to a curvilinear articulating arm;
fixing the curvilinear articulating arm in a position so that the desired pressure is maintained proximate the puncture without continuing to manually apply the desired pressure.

15. The method of claim 14, further comprising:
monitoring the puncture using ultrasound imaging, wherein the compression member comprises an ultrasound transducer.

16. The method of claim 15, wherein the ultrasound transducer is disposed in a sterile sheath.

17. The method of claim 15, wherein the ultrasound transducer is separated from the skin by a separate layer of silicone.

18. The method of claim 14, further comprising:
monitoring the desired pressure.

19. The method of claim 14, further comprising:
measuring the desired pressure.

20. The method of claim 14, wherein the desired pressure is applied by the compression member prior to coupling the compression member to the curvilinear articulating arm.

21. A method for vascular tamponade comprising:
manually applying a desired pressure proximate a puncture in a vessel by positioning a compression member to apply pressure against skin of a patient, the desired pressure permitting clot formation at the puncture;
coupling the compression member to an object disposed in fixed relationship to the puncture;
fixing the compression member in a position so that the desired pressure is maintained proximate the puncture without continuing to manually apply the desired pressure.

22. The method of claim 21, wherein the object is a rail associated with a bed.
